(19) United States
(12) Patent Application Publication
(10) Pub. No.: US 2024/0153598 A1
(43) Pub. Date: May 9, 2024

(54) SYSTEMS AND METHODS FOR CONTENT CUSTOMIZATION

(71) Applicant: ADOBE INC., SAN JOSE, CA (US)

(72) Inventors: Raghavendra Kiran Addanki, Santa Clara, CA (US); David Arbour, Charlottesville, VA (US); Tung Mai, San Jose, CA (US); Anup Bandigadi Rao, San Jose, CA (US); Cameron N. Musco, Hadley, MA (US)

(21) Appl. No.: 18/051,736

(22) Filed: Nov. 1, 2022

(51) Int. Cl. G16H 10/20

(52) U.S. Cl. CPC G16H 10/20

(57) ABSTRACT

Systems and methods for content customization are described. According to one aspect, a content customization apparatus is provided. The apparatus includes a processor; a memory storing instructions executable by the processor; a user feature component configured to generate user feature vectors representing user features for a plurality of users, respectively; a group selection component configured to select a treatment group and a control group based on the user feature vectors; a machine learning model configured to train a treatment effect estimator based on the user feature vectors and outcome data for the treatment group and the control group; and a content component configured to provide customized content based on the treatment effect estimator.

### JOINT RESEARCH AGREEMENT

The presently claimed invention was made by or on behalf of the below listed parties to a joint research agreement. The joint research agreement was in effect on or before the date the claimed invention was made, and the claimed invention was part of the joint research agreement and made as a result of activities undertaken within the scope of the joint research agreement. The parties to the joint research agreement are Adobe Inc. and the University of Massachusetts.

### BACKGROUND

The following relates to content customization based on an estimation of a treatment effect. Treatment effect determination is a fundamental aspect of causal inference. To determine an effect that a treatment (i.e., a stimulus) may have on a given population, a subset of the population may be assigned to a treatment group and a subset of the population may be assigned to a control group. The treatment may be provided to the treatment group, and a different treatment, a placebo, or no treatment may be provided to the control group. A difference in reaction between the treatment group and the control group (i.e., a treatment effect estimator) may be observed. A treatment effect for the general population is estimated based on the treatment effect estimator.

However, conventional treatment effect estimation techniques use large sample sizes and are prone to inaccuracy due to uniform sampling. Therefore, there is a need in the art for systems and methods that minimize a size of the treatment group and the control group and reduce error in an estimated treatment effect resulting from a sampling process.

### SUMMARY

Embodiments of the present disclosure determine a treatment group and a control group from among a set of users based on a set of user feature vectors for the set of users, thereby minimizing a size of the treatment group and the control group, which minimizes costs associated with running a treatment experiment. Embodiments of the present disclosure use a machine learning model to train a treatment effect estimator based on outcome data and the set of user feature vectors, thereby increasing an accuracy of the treatment effect estimator.

Embodiments of the present disclosure provide customized content to a user based on the treatment effect estimator, thereby allowing targeted content to be provided to the user based on a more accurate estimate that the user will take an action in response to receiving the targeted content than conventional systems provide.

A method, apparatus, non-transitory computer readable medium, and system for content customization are described. One or more aspects of the method, apparatus, non-transitory computer readable medium, and system include identifying a user feature matrix that represents user features for each of a plurality of users; computing a leverage score for each of the plurality of users based on the user feature matrix; generating a treatment sampling matrix for a treatment group and a control sampling matrix for a control group based on the leverage score; training an individual treatment effect estimator using a machine learning model based on the treatment sampling matrix, the control sampling matrix, the user feature matrix, and outcome data for the plurality of users; and providing customized content for a user based on the individual treatment effect estimator.

A method, apparatus, non-transitory computer readable medium, and system for content customization are described. One or more aspects of the method, apparatus, non-transitory computer readable medium, and system include identifying a plurality of feature vectors that represent user features for a plurality of users, respectively; generating a treatment group and a control group from the plurality of users by recursively partitioning the plurality of users based on the plurality of feature vectors; training an average treatment effect estimator using a machine learning model based on outcome data for the treatment group and the control group; and providing customized content for a user based on the average treatment effect estimator.

An apparatus and system for content customization are described. One or more aspects of the apparatus and system include a processor; a memory storing instructions executable by the processor; a user feature component configured to generate user feature vectors representing user features for a plurality of users, respectively; a group selection component configured to select a treatment group and a control group based on the user feature vectors; a machine learning model configured to train a treatment effect estimator based on the user feature vectors and outcome data for the treatment group and the control group; and a content component configured to provide customized content based on the treatment effect estimator.

### BRIEF DESCRIPTION OF THE DRAWINGS

FIG. 1 shows an example of a content customization system according to aspects of the present disclosure.

FIG. 2 shows an example of a content customization apparatus according to aspects of the present disclosure.

FIG. 3 shows a first example of data flow in a content customization apparatus according to aspects of the present disclosure.

FIG. 4 shows a second example of data flow in a content customization apparatus according to aspects of the present disclosure.

FIG. 5 shows an example of content customization according to aspects of the present disclosure.

FIG. 6 shows an example of customizing content based on an individual treatment effect estimator according to aspects of the present disclosure.

FIG. 7 shows an example of an algorithm for determining an individual treatment effect estimator according to aspects of the present disclosure.

FIG. 8 shows an example of customizing content based on an average treatment effect estimator according to aspects of the present disclosure.

FIG. 9 shows an example of partitioning a set of users according to aspects of the present disclosure.

FIG. 10 shows an example of an algorithm for determining an average treatment effect estimator according to aspects of the present disclosure.

### DETAILED DESCRIPTION

Embodiments of the present disclosure relate to content customization based on an estimation of a treatment effect. Treatment effect determination is a fundamental aspect of causal inference. To determine an effect that a treatment (i.e., a stimulus) may have on a given population, a subset of the population may be assigned to a treatment group and a subset of the population may be assigned to a control group. The treatment may be provided to the treatment group, and a different treatment, a placebo, or no treatment may be provided to the control group. A difference in reaction between the treatment group and the control group (i.e., a treatment effect estimator) may be observed. A treatment effect for the general population is estimated based on the treatment effect estimator.

However, conventional treatment effect estimation techniques use large sample sizes and are prone to inaccuracy due to uniform sampling. For example, conventional treatment effect estimation techniques either randomly divide an entire population into a treatment group and a control group, which is unfeasibly expensive at scale, or employ uniform sampling to uniformly select a subset of the population, and then randomly assign members of the subset to a treatment group and a control group. Uniform sampling is not robust to outliers and results in an inaccurate treatment effect estimate. Randomly assigning members of the subset to the treatment group and the control group introduces further error in the treatment effect estimate when the distribution of randomly assigned members in the groups is not representative of the entire population.

Accordingly, aspects of the present disclosure identify a treatment group and a control group from a set of users based on user feature vectors for the set of users, use a machine learning model to train a treatment effect estimator based on outcome data and the set of user feature vectors, and provide customized content to a user based on the treatment effect estimator.

By determining the treatment group and the control group based on the set of user feature vectors, embodiments of the present disclosure minimize a size of the treatment group and the control group, thereby minimizing costs associated with running a treatment experiment. By determining the treatment effect estimator based on the outcome data and the set of user feature vectors, embodiments of the present disclosure increase an accuracy of the treatment effect estimator. Therefore, by providing customized content based on the treatment effect estimator, aspects of the present disclosure allow targeted content to be provided to the user based on a more accurate estimate that the user will take an action in response to receiving the targeted content than conventional systems provide. This approach to content customization optimizes an allocation of resources by delivering customized content to users who will likely respond in an expected manner to the customized content, and by avoiding delivering customized content to users who will not respond in the expected manner.

According to an aspect of the present disclosure, a content customization system is provided. According to some aspects, the content customization system includes a user feature component, a group selection component, a machine learning model, and a content component.

In some cases, the user feature component generates user feature vectors representing user features for a set of users, respectively. In some cases, the group selection component selects a treatment group and a control group based on the user feature vectors. In some cases, the machine learning model trains a treatment effect estimator based on the user feature vectors and outcome data for the treatment group and the control group. In some cases, the content component provides customized content based on the treatment effect estimator.

Embodiments of the present disclosure include individual and average treatment effect estimation under a linear effects model. In some cases, efficient experimental designs and corresponding estimators are provided by identifying connections to discrepancy minimization and leverage-score-based sampling used in randomized numerical linear algebra.

In some cases, the user feature component identifies a user feature matrix that represents user features for each of a set of users. In some cases, the group selection component computes a leverage score for each of the set of users based on the user feature matrix. In some cases, the group selection component generates a treatment sampling matrix for a treatment group and a control sampling matrix for a control group based on the leverage score. In some cases, the machine learning model trains an individual treatment effect estimator based on the treatment sampling matrix, the control sampling matrix, the user feature matrix, and outcome data for the set of users. In some cases, the content component provides customized content for a user based on the individual treatment effect estimator.

By generating the treatment sampling matrix based on the leverage score, the system identifies a set of users for inclusion in a treatment group and a control group who are statistically important (i.e., users that are representative of the set of users as a whole), thereby enabling the system to minimize a size of the treatment group and the control group and costs associated with determining whether the customized content is projected to be effective.

In some cases, the user feature component identifies a set of feature vectors that represent user features for a set of users, respectively. In some cases, the group selection component generates a treatment group and a control group from the set of users by recursively partitioning the set of users based on the set of feature vectors. In some cases, the machine learning model trains an average treatment effect estimator based on outcome data for the treatment group and the control group. In some cases, the content component provides customized content for a user based on the average treatment effect estimator.

By recursively partitioning the set of users based on the user feature vectors, the system minimizes a size of the treatment group and the control group and costs associated with determining whether the customized content is projected to be effective. Furthermore, by determining the average treatment effect estimator based on outcome data derived from the recursive partitioning, the system increases an accuracy of the average treatment effect estimator compared to an average treatment effect estimator that is derived from a uniform sampling process.

As used herein, a “user feature” refers to data that is characteristic of a user. Examples of a user feature include a user identifier, user profile data associated with the user, a user device identifier, a location of the user, a location of the user device, demographic information of the user, data that is produced by an interaction of the user and/or the user device with another entity, such as a third party user or organization, or another user, etc. In some cases, the interaction occurs via a content channel such a website, a software application, a messaging service, an email service, a physical location such as a store, restaurant, hotel, etc., or a combination thereof. In some cases, data that is produced by the interaction includes a type of the interaction (such as a website visit, a hyperlink click, an addition or removal of an item to or from a digital shopping cart, a purchase, a check-in, a presence of a user device within a geofenced area, etc.), a time of the interaction, a location of the interaction, an identification of a web browser or other software corresponding to the interaction, an identification of another entity corresponding to the interaction, etc.

As used herein, a “user feature matrix” includes a set of rows, wherein each row of the set of rows includes a user feature vector that is a representation of user features for a user of a set of users. As used herein, a “leverage score” refers to a numerical representation of an importance of a row corresponding to a user to a row space of the user feature matrix. For example, a leverage score measures an importance of an individual user to a set of users based on the user's user feature vectors (e.g., covariates). As used herein, a “sampling matrix” refers to a matrix including user feature vectors that are sampled form the user feature matrix.

As used herein, a “treatment group” refers to a group of users among a set of users who are identified to receive customized content. As used herein, a “control group” refers to a group of users among the set of users who are identified to not receive the customized content. As used herein, “customized content” refers to content (including media, such as text media, image media, video media, audio media, or a combination thereof; a message; an email; a website; a weblink; a software application; etc.) that is targeted based on user feature data. In an example, user feature data can include a location of user in California and an identification of a smartphone as a user device, and the customized content can be targeted to appeal to users from California who use smartphones.

As used herein, “outcome data” refers to data produced by an action of a user. In some cases, the outcome data relates to a response to some stimulus (such as receiving the customized content). For example, a user may take some action in response to receiving the customized content (such as clicking on a weblink, making a purchase, making a communication, etc.), and the action produces data relating to the action.

As used herein, a “treatment effect” refers to a numerical representation of a difference in outcome data for the treatment group and the control group. As used herein, a “treatment effect estimator” refers to a function that estimates a value for a treatment effect. As used herein, an “individual treatment effect estimator” refers to a function that estimates a treatment effect for an individual user. As used herein, an “average treatment effect estimator” refers to an estimate of a treatment effect for a group of users.

An embodiment of the present disclosure is used in an A/B testing context. In an example, a third-party user wants to determine an effect that website A has on a set of users as compared to website B. In some cases, website A is tailored to appeal to users in a particular demographic, as reflected in user features for the users in the particular demographic. In some cases, the third-party user provides website A (e.g., the customized content) and website B to a content customization apparatus.

In some cases, a user feature component of the content customization apparatus identifies a user feature matrix that includes vector representations of user features for each of the set of users, where each user corresponds to a row of the user feature matrix.

In some cases, a group selection component of the content customization computes a leverage score for each of the set of users based on the user feature matrix. In some cases, the leverage score identifies an importance of each row of the user feature matrix (and a corresponding user) to the embedding space of the user feature matrix, thereby allowing the content customization apparatus to numerically identify users that will tend to be representative of the set of users as a whole.

In some cases, the group selection component generates a treatment sampling matrix for a treatment group and a control sampling matrix for a control group based on the leverage score. In some cases, the group selection component identifies the treatment group using the treatment sampling matrix and identifies the control group using the control sampling matrix. By deriving the treatment group and the control group via the leverage score sampling process, the system minimizes a number of users who are included in either the treatment group or the control group, thereby minimizing a cost of running the A/B test.

In some cases, a content component of the content customization apparatus provides website A to users in the treatment group via a content channel, provides website B to users in the control group via the content channel, and monitors the content channel (for example, via API calls) to determine whether users in the treatment group and the control group click on similar areas of website A and website B, respectively. The content component thereby obtains outcome data for the treatment group and the control group.

In some cases, a machine learning model of the content customization apparatus trains an individual treatment effect estimator based on the treatment sampling matrix, the control sampling matrix, the user feature matrix, and the outcome data, thereby minimizing an error in the individual treatment effect estimator.

In some cases, the content component provides website A to a user based on the individual treatment effect estimator. For example, the content component evaluates the individual treatment effect estimator for the user and determines that a resulting treatment effect estimation exceeds a treatment effect threshold. Based on the determination, the content component identifies that the user is likely to take a desired action of clicking on a targeted area of website A, and therefore provides website A to the user.

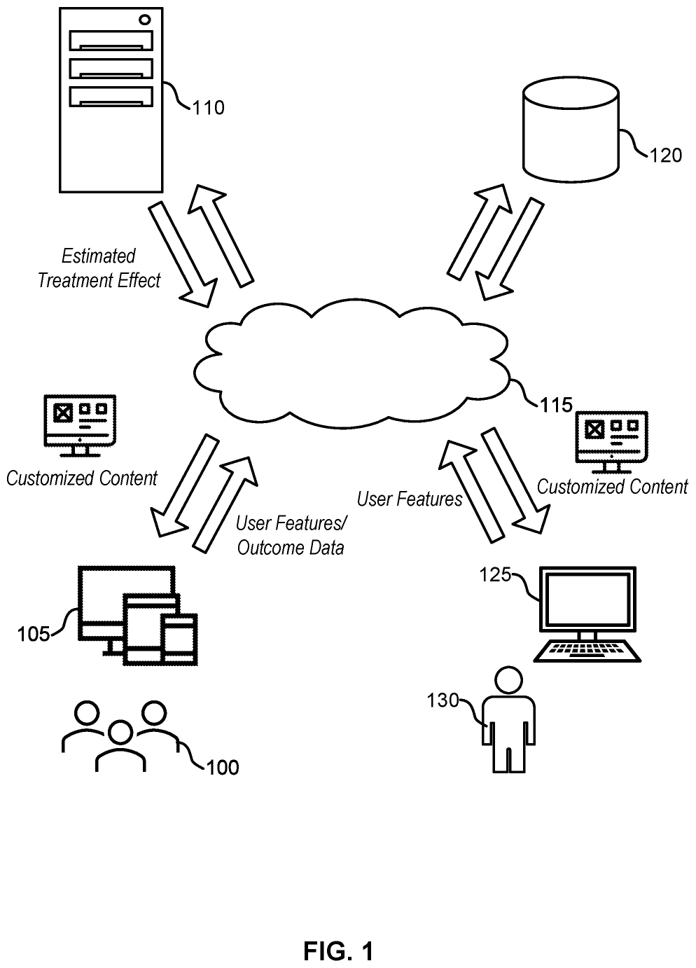

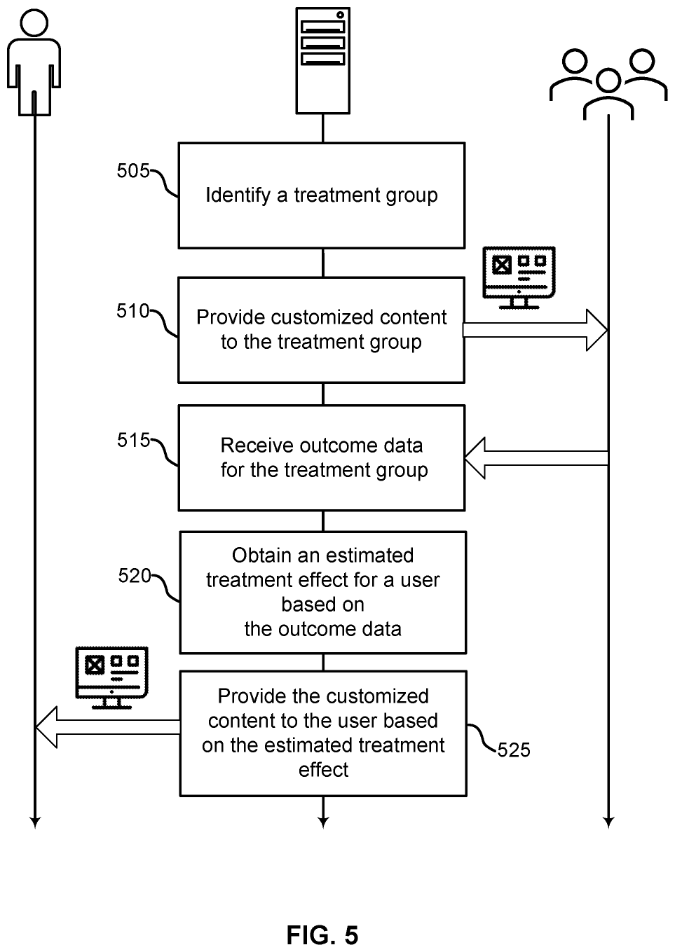

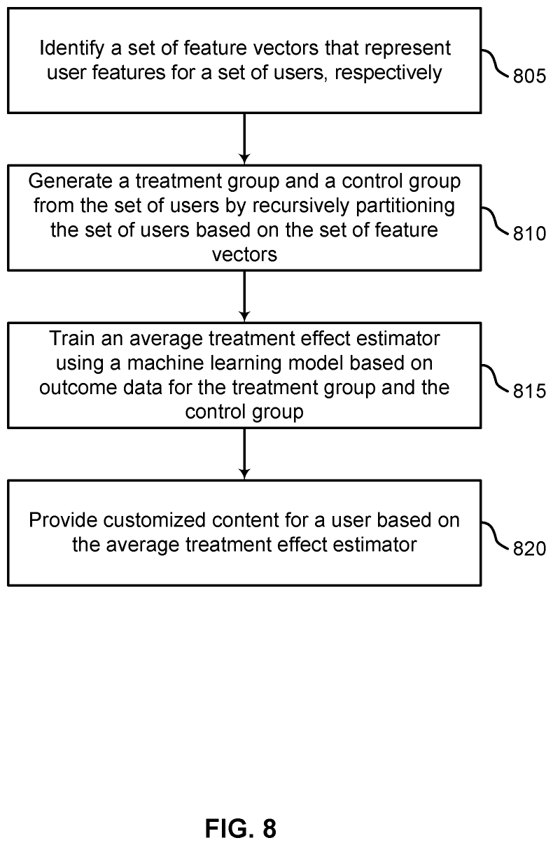

Example applications of the present disclosure in an A/B testing context are provided with reference to FIGS. 1 and 5. Details regarding the architecture of the content customization apparatus process are provided with reference to FIGS. 1-4. Examples of a process for content customization based on an individual treatment effect estimator are provided with reference to FIGS. 5-7. Examples of a process for content customization based on an average treatment effect estimator are provided with reference to FIGS. 8-10.

### Content Customization System

An apparatus and system for content customization is described with reference to FIGS. 1-4. One or more aspects of the apparatus and system include a processor; a memory storing instructions executable by the processor; a user feature component configured to generate user feature vectors representing user features for a plurality of users, respectively; a group selection component configured to select a treatment group and a control group based on the user feature vectors; a machine learning model configured to train a treatment effect estimator based on the user feature vectors and outcome data for the treatment group and the control group; and a content component configured to provide customized content based on the treatment effect estimator.

In some aspects, the group selection component is further configured to generate a selection probability function for each of the plurality of users, wherein the treatment group and the control group are selected based on the selection probability function. In some aspects, the group selection component is further configured to identify a user in the treatment group and the control group and to remove the user from the treatment group or the control group.

In some aspects, the group selection component is further configured to recursively partition the plurality of users based on the user feature vectors, wherein the treatment group and the control group are selected based on the partitioning. In some aspects, the partitioning is based on a Gram-Schmidt-Walk algorithm. In some aspects, the group selection component is further configured to identify pairs of similar users among the plurality of users and to select a user from each of the pairs, wherein the partitioning is based on the selected user.

FIG. 1 shows an example of a content customization system according to aspects of the present disclosure. The example shown includes treatment group 100, treatment group user devices 105, content customization apparatus 110, cloud 115, database 120, user device 125, and user 130.

Referring to FIG. 1, content customization apparatus 110 receives user features from treatment group 100 and user 130 via treatment group user devices 105 and user device 125, respectively, and identifies treatment group 100 based on the user features. Content customization apparatus 110 provides customized content to treatment group 100 and receives outcome data from treatment group 100 in response. Content customization apparatus 110 determines an estimated treatment effect based on the outcome data and provides the customized content to user 130 based on the estimated treatment effect.

According to some aspects, each of treatment group user devices 105 and user device 125 is a personal computer, laptop computer, mainframe computer, palmtop computer, personal assistant, mobile device, or any other suitable processing apparatus. In some examples, each of treatment group user devices 105 and user device 125 includes software that allows users in treatment group 100 and user 130 to respectively interact with other entities (such as other users, a web browser, an app, a content channel, etc.) and to receive content from content customization apparatus 110.

According to some aspects, separate user interfaces enable users in treatment group 100 and user 130 to respectively interact with treatment group user devices 105 and user device 125. In some embodiments, each of the separate user interfaces include an audio device, such as an external speaker system, an external display device such as a display screen, or an input device (e.g., a remote control device interfaced with the user interface directly or through an IO controller module). In some cases, each of the separate user interfaces is a graphical user interface (GUI).

According to some aspects, content customization apparatus 110 includes a computer implemented network. In some embodiments, the computer implemented network includes an artificial neural network (ANN). In some embodiments, content customization apparatus 110 also includes one or more processors, a memory subsystem, a communication interface, an I/O interface, one or more user interface components, and a bus. Additionally, in some embodiments, content customization apparatus 110 communicates with treatment group user devices 105, database 120, user device 125, or a combination thereof via cloud 115.

In some cases, content customization apparatus 110 is implemented on a server. A server provides one or more functions to users linked by way of one or more of various networks, such as cloud 115. In some cases, the server includes a single microprocessor board, which includes a microprocessor responsible for controlling all aspects of the server. In some cases, the server uses microprocessor and protocols to exchange data with other devices or users on one or more of the networks via hypertext transfer protocol (HTTP), and simple mail transfer protocol (SMTP), although other protocols such as file transfer protocol (FTP), and simple network management protocol (SNMP) may also be used. In some cases, the server is configured to send and receive hypertext markup language (HTML) formatted files (e.g., for displaying web pages). In various embodiments, the server comprises a general purpose computing device, a personal computer, a laptop computer, a mainframe computer, a supercomputer, or any other suitable processing apparatus.

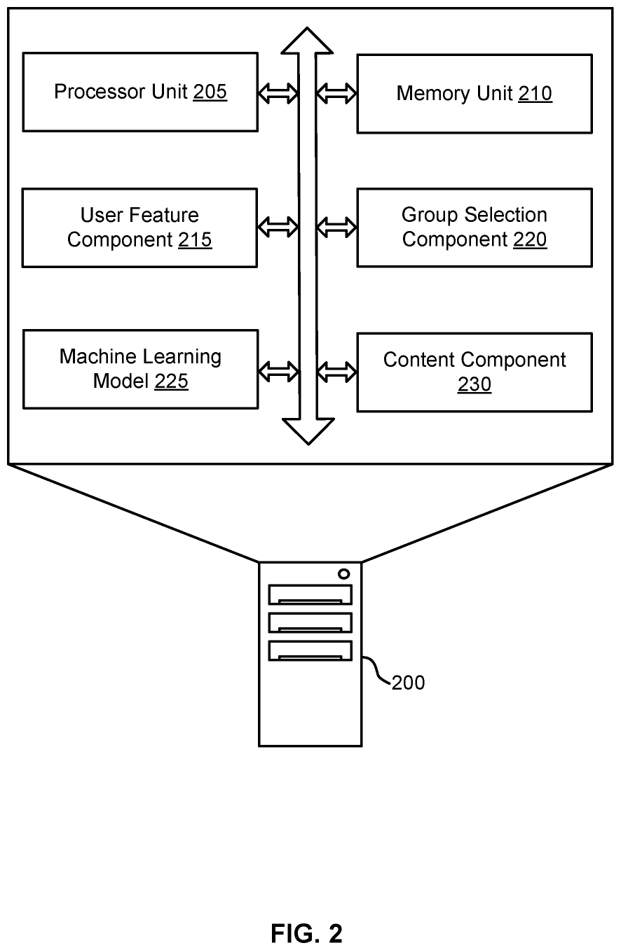

Further details regarding the architecture of content customization apparatus 110 are provided with reference to FIGS. 2-4. Further detail regarding a process for content customization based on an individual treatment effect estimator is provided with reference to FIGS. 5-7. Further detail regarding a process for content customization based on an average treatment effect estimator is provided with reference to FIGS. 8-10.

Cloud 115 is a computer network configured to provide on-demand availability of computer system resources, such as data storage and computing power. In some examples, cloud 115 provides resources without active management by a user. The term “cloud” is sometimes used to describe data centers available to many users over the Internet. Some large cloud networks have functions distributed over multiple locations from central servers. A server is designated an edge server if it has a direct or close connection to a user. In some cases, cloud 115 is limited to a single organization. In other examples, cloud 115 is available to many organizations. In one example, cloud 115 includes a multi-layer communications network comprising multiple edge routers and core routers. In another example, cloud 115 is based on a local collection of switches in a single physical location. According to some aspects, cloud 115 provides communications between treatment group user devices 105, content customization apparatus 110, database 120, user device 125, or a combination thereof.

Database 120 is an organized collection of data. In an example, database 120 stores data in a specified format known as a schema. According to some aspects, database 120 is structured as a single database, a distributed database, multiple distributed databases, or an emergency backup database. In some cases, a database controller manages data storage and processing in database 120. In some cases, a user interacts with the database controller. In other cases, the database controller operates automatically without interaction from the user. In some aspects, database 120 is external to content customization apparatus 110 and communicates with content customization apparatus 110 via cloud 115. In some embodiments, database 120 is included in content customization apparatus 110.

FIG. 2 shows an example of a content customization apparatus according to aspects of the present disclosure. According to some aspects, content customization apparatus 200 includes processor unit 205, memory unit 210, user feature component 215, group selection component 220, machine learning model 225, and content component 230.

According to some aspects, processor unit 205 includes one or more processors. A processor is an intelligent hardware device, such as a general-purpose processing component, a digital signal processor (DSP), a central processing unit (CPU), a graphics processing unit (GPU), a microcontroller, an application specific integrated circuit (ASIC), a field programmable gate array (FPGA), a programmable logic device, a discrete gate or transistor logic component, a discrete hardware component, or any combination thereof. In some cases, processor unit 205 is configured to operate a memory array using a memory controller. In other cases, a memory controller is integrated into processor unit 205. In some cases, processor unit 205 is configured to execute computer-readable instructions stored in memory unit 210 to perform various functions. In some embodiments, processor unit 205 includes special purpose components for modem processing, baseband processing, digital signal processing, or transmission processing.

According to some aspects, memory unit 210 includes one or more memory devices. Examples of a memory device include random access memory (RAM), read-only memory (ROM), or a hard disk. Examples of memory devices include solid state memory and a hard disk drive. In some examples, memory is used to store computer-readable, computer-executable software including instructions that, when executed, cause a processor of processor unit 205 to perform various functions described herein. In some cases, memory unit 210 includes a basic input/output system (BIOS) that controls basic hardware or software operations, such as an interaction with peripheral components or devices. In some cases, memory unit 210 includes a memory controller that operates memory cells of memory unit 210. For example, the memory controller may include a row decoder, column decoder, or both. In some cases, memory cells within memory unit 210 store information in the form of a logical state.

According to some aspects, user feature component 215 identifies a user feature matrix that represents user features for each of a set of users. According to some aspects, user feature component 215 identifies a set of feature vectors that represent user features for a set of users, respectively. In some examples, user feature component 215 performs a smoothing operation on the user feature matrix to obtain a smoothed user feature matrix, where the treatment sampling matrix is based on the smoothed user feature matrix.

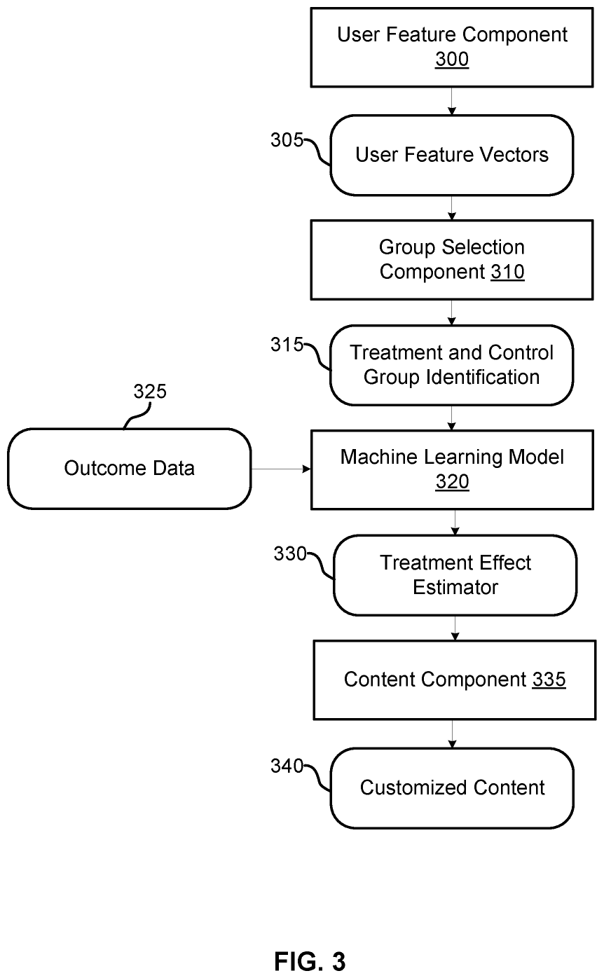

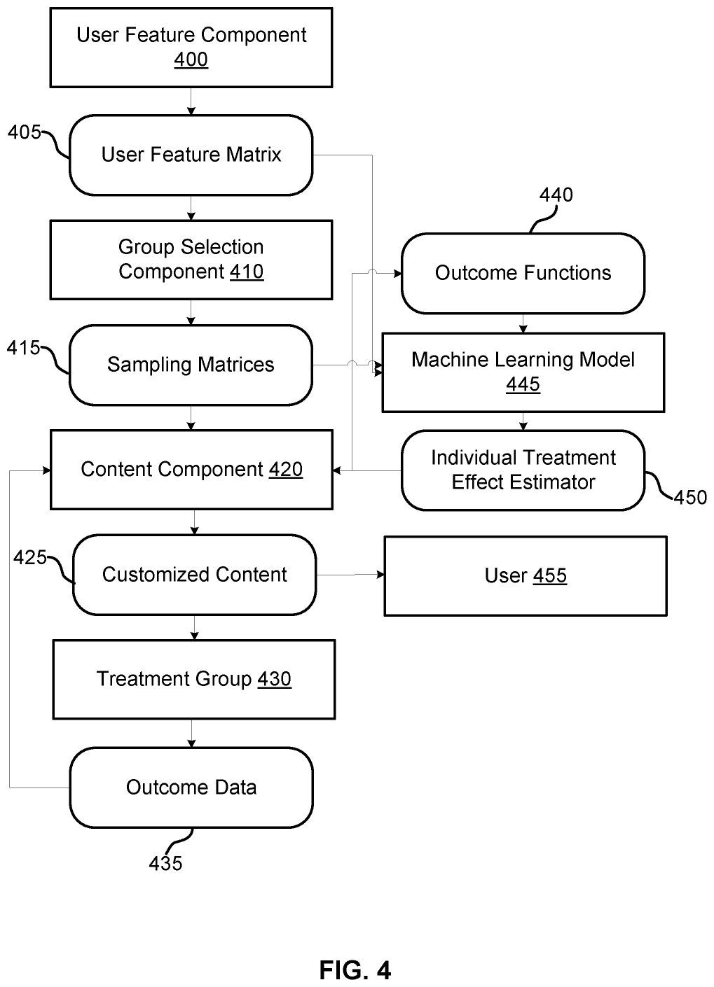

According to some aspects, user feature component 215 is configured to generate user feature vectors representing user features for a plurality of users, respectively. User feature component 215 is an example of, or includes aspects of, the corresponding element described with reference to FIGS. 3-4. According to some aspects, user feature component 215 is implemented as one or more hardware circuits, as firmware, as software stored in memory of memory unit 210 and executed by a processor of processor unit 205, or as a combination thereof.

According to some aspects, group selection component 220 computes a leverage score for each of the set of users based on the user feature matrix. In some examples, group selection component 220 generates a treatment sampling matrix for a treatment group and a control sampling matrix for a control group based on the leverage score.

In some examples, group selection component 220 generates a selection probability function based on the leverage score for each of the set of users. In some examples, group selection component 220 selects the treatment group and the control group based on the selection probability function.

In some examples, group selection component 220 identifies a user in the treatment group and the control group. In some examples, group selection component 220 removes the user from the treatment group or the control group.

According to some aspects, group selection component 220 generates a treatment group and a control group from the set of users by recursively partitioning the set of users based on the set of feature vectors. In some examples, group selection component 220 partitions the set of users based on a Gram-Schmidt-Walk algorithm.

In some examples, group selection component 220 identifies pairs of similar users among the set of users. In some examples, group selection component 220 selects a user from each of the pairs, where the partitioning is based on the selected user. In some examples, group selection component 220 identifies a size for the treatment group. In some examples, group selection component 220 selects a number of iterations based on the size, where the partitioning is based on the number of iterations. In some aspects, the treatment group includes a coreset of the set of users.

According to some aspects, group selection component 220 is configured to select a treatment group and a control group based on the user feature vectors. In some aspects, group selection component 220 is further configured to generate a selection probability function for each of the set of users, where the treatment group and the control group are selected based on the selection probability function. In some aspects, group selection component 220 is further configured to identify a user in the treatment group and the control group and to remove the user from the treatment group or the control group.

In some aspects, group selection component 220 is further configured to recursively partition the set of users based on the user feature vectors, where the treatment group and the control group are selected based on the partitioning. In some aspects, the partitioning is based on a Gram-Schmidt-Walk algorithm. In some aspects, group selection component 220 is further configured to identify pairs of similar users among the set of users and to select a user from each of the pairs, where the partitioning is based on the selected user.

Group selection component 220 is an example of, or includes aspects of, the corresponding element described with reference to FIGS. 3-4. According to some aspects, group selection component 220 is implemented as one or more hardware circuits, as firmware, as software stored in memory of memory unit 210 and executed by a processor of processor unit 205, or as a combination thereof.

According to some aspects, machine learning model 225 trains an individual treatment effect estimator based on the treatment sampling matrix, the control sampling matrix, the user feature matrix, and outcome data for the set of users. In some aspects, the machine learning model 225 includes a regression on a treatment outcome function and a control outcome function, where the individual treatment effect estimator is based on the treatment outcome function and the control outcome function. According to some aspects, machine learning model 225 trains an average treatment effect estimator based on outcome data for the treatment group and the control group.

According to some aspects, machine learning model 225 is configured to train a treatment effect estimator based on the user feature vectors and outcome data for the treatment group and the control group. According to some aspects, machine learning model 225 includes one or more artificial neural networks (ANNs). An ANN is a hardware or a software component that includes a number of connected nodes (i.e., artificial neurons) that loosely correspond to the neurons in a human brain. Each connection, or edge, transmits a signal from one node to another (like the physical synapses in a brain). When a node receives a signal, it processes the signal and then transmits the processed signal to other connected nodes. In some cases, the signals between nodes comprise real numbers, and the output of each node is computed by a function of the sum of its inputs. In some examples, nodes may determine their output using other mathematical algorithms, such as selecting the max from the inputs as the output, or any other suitable algorithm for activating the node. Each node and edge are associated with one or more node weights that determine how the signal is processed and transmitted.

In ANNs, a hidden (or intermediate) layer includes hidden nodes and is located between an input layer and an output layer. Hidden layers perform nonlinear transformations of inputs entered into the network. Each hidden layer is trained to produce a defined output that contributes to a joint output of the output layer of the neural network. Hidden representations are machine-readable data representations of an input that are learned from a neural network's hidden layers and are produced by the output layer. As the neural network's understanding of the input improves as it is trained, the hidden representation is progressively differentiated from earlier iterations.

During a training process of an ANN, the node weights are adjusted to improve the accuracy of the result (i.e., by minimizing a loss which corresponds in some way to the difference between the current result and the target result). The weight of an edge increases or decreases the strength of the signal transmitted between nodes. In some cases, nodes have a threshold below which a signal is not transmitted at all. In some examples, the nodes are aggregated into layers. Different layers perform different transformations on their inputs. The initial layer is known as the input layer and the last layer is known as the output layer. In some cases, signals traverse certain layers multiple times.

According to some aspects, machine learning model comprises a regression ANN. Machine learning model 225 is an example of, or includes aspects of, the corresponding element described with reference to FIGS. 3-4. According to some aspects, machine learning model 225 is implemented as one or more hardware circuits, as firmware, as software stored in memory of memory unit 210 and executed by a processor of processor unit 205, or as a combination thereof.

According to some aspects, content component 230 provides customized content for a user based on the individual treatment effect estimator. In some examples, content component 230 provides the customized content to the treatment group. In some examples, content component 230 monitors an outcome after providing the customized content, where the outcome data is obtained based on the monitoring. In some examples, content component 230 computes an estimated treatment effect for the user based on the individual treatment effect estimator. In some examples, content component 230 determines to provide the customized content to the user based on the estimated treatment effect.

According to some aspects, content component 230 provides customized content for a user based on the average treatment effect estimator. In some examples, content component 230 provides the customized content to the treatment group. In some examples, content component 230 monitors an outcome after providing the customized content, where the outcome data is obtained based on the monitoring. In some examples, content component 230 computes an estimated treatment effect for the user based on the average treatment effect estimator. In some examples, content component 230 determines to provide the customized content to the user based on the estimated treatment effect.

According to some aspects, content component 230 is configured to provide customized content based on the treatment effect estimator. Content component 230 is an example of, or includes aspects of, the corresponding element described with reference to FIGS. 3-4. According to some aspects, content component 230 is implemented as one or more hardware circuits, as firmware, as software stored in memory of memory unit 210 and executed by a processor of processor unit 205, or as a combination thereof.

FIG. 3 shows a first example of data flow in a content customization apparatus according to aspects of the present disclosure. The example shown includes user feature component 300, user feature vectors 305, group selection component 310, treatment and control group identification 315, machine learning model 320, outcome data 325, treatment effect estimator 330, content component 335, and customized content 340.

User feature component 300, group selection component 310, machine learning model 320, and content component 335 are examples of, or include aspects of, the corresponding elements respectively described with reference to FIGS. 2 and 4. Outcome data 325 and customized content 340 are examples of, or include aspects of, the corresponding elements described with reference to FIG. 4.

Referring to FIG. 3, user feature component 300 generates user feature vectors 305 representing user features for a plurality of users, respectively. Group selection component 310 determines treatment and control group identification 315 based on user feature vectors 305. Machine learning model 320 trains treatment effect estimator 330 based on treatment and control group identification 315 and outcome data 325 for the treatment group and the control group. Content component 335 determines that customized content 340 is to be provided to a user based on treatment effect estimator 330.

FIG. 4 shows a second example of data flow in a content customization system according to aspects of the present disclosure. The example shown includes user feature component 400, user feature matrix 405, group selection component 410, sampling matrices 415, content component 420, customized content 425, treatment group 430, outcome data 435, outcome functions 440, machine learning model 445, individual treatment effect estimator 450, and user 455.

Treatment group 430 and user 455 are examples of, or include aspects of, the corresponding elements described with reference to FIG. 1. User feature component 400, group selection component 410, content component 420, and machine learning model 445 are examples of, or include aspects of, the corresponding elements respectively described with reference to FIGS. 2 and 3. Outcome data 435 and customized content 425 are examples of, or include aspects of, the corresponding elements described with reference to FIG. 4.

Referring to FIG. 4, user feature component 400 provides user feature matrix 405 to group selection component 410. In some cases, user feature matrix 405 is a smoothed user feature matrix. Group selection component 410 generates sampling matrices 415 for treatment group 430 and a control group based on user feature matrix 405. Content component 420 receives sampling matrices 415 from group selection component 410 and provides customized content 425 to treatment group 430 based on sampling matrices 415.

Treatment group 430 provides outcome data 435 to content component 420. Content component 420 determines outcome functions 440 based on outcome data 435, and provides outcome functions 440 to machine learning model 445. Machine learning model 445 receives user feature matrix 405 from user feature component 400 and receives sampling matrices 415 from group selection component 410.

Machine learning model 445 trains individual treatment effect estimator 450 based on user feature matrix 405, sampling matrices 415, and outcome functions 440. Machine learning model 445 provides individual treatment effect estimator 450 to content component 420. Content component 420 provides customized content 425 to user 455 based on individual treatment effect estimator 450.

### Customizing Content Based on an Individual Treatment Effect Estimator

A method for content customization is described with reference to FIGS. 5-7. One or more aspects of the method include identifying a user feature matrix that represents user features for each of a plurality of users; computing a leverage score for each of the plurality of users based on the user feature matrix; generating a treatment sampling matrix for a treatment group and a control sampling matrix for a control group based on the leverage score; training an individual treatment effect estimator using a machine learning model based on the treatment sampling matrix, the control sampling matrix, the user feature matrix, and outcome data for the plurality of users; and providing customized content for a user based on the individual treatment effect estimator.

Some examples of the method further include performing a smoothing operation on the user feature matrix to obtain a smoothed user feature matrix, wherein the treatment sampling matrix is based on the smoothed user feature matrix. Some examples of the method further include generating a selection probability function based on the leverage score for each of the plurality of users. Some examples further include selecting the treatment group and the control group based on the selection probability function.

Some examples of the method further include identifying a user in the treatment group and the control group. Some examples further include removing the user from the treatment group or the control group. Some examples of the method further include providing the customized content to the treatment group. Some examples further include monitoring an outcome after providing the customized content, wherein the outcome data is obtained based on the monitoring.

In some aspects, the machine learning model comprises a regression on a treatment outcome function and a control outcome function, wherein the individual treatment effect estimator is based on the treatment outcome function and the control outcome function.

Some examples of the method further include computing an estimated treatment effect for the user based on the individual treatment effect estimator. Some examples further include determining to provide the customized content to the user based on the estimated treatment effect.

FIG. 5 shows an example of content customization according to aspects of the present disclosure. In some examples, these operations are performed by a system including a processor executing a set of codes to control functional elements of an apparatus. Additionally or alternatively, certain processes are performed using special-purpose hardware. Generally, these operations are performed according to the methods and processes described in accordance with aspects of the present disclosure. In some cases, the operations described herein are composed of various substeps, or are performed in conjunction with other operations.

Referring to FIG. 5, according to some aspects, a content customization apparatus identifies a treatment group of a set of users based on user feature data. The content customization apparatus provides customized content (e.g., content that is tailored for users that correspond to certain user features) to the treatment group. By determining the treatment group based on the user features, the content customization system minimizes a number of users in the treatment group. The content customization apparatus receives outcome data from the treatment group in response to the customized content. The content customization apparatus determines an estimated treatment effect for a user based on the outcome data. The content customization apparatus provides the customized content to the user based on the estimated treatment effect.

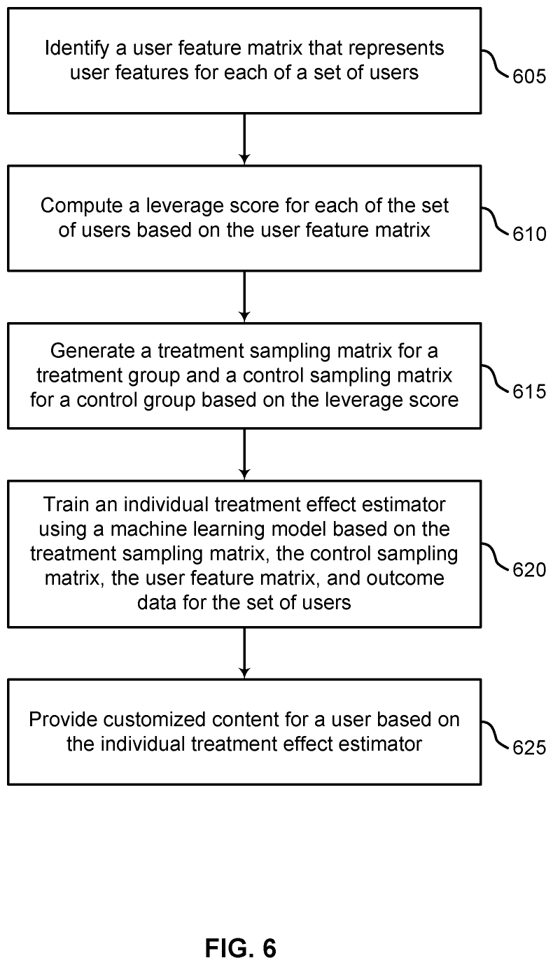

At operation 505, the system identifies a treatment group. In some cases, the operations of this step refer to, or may be performed by, a content customization apparatus as described with reference to FIGS. 1-2. For example, in some cases, the content customization apparatus identifies the treatment group based on a user feature matrix as described with reference to FIG. 6.

At operation 510, the system provides customized content to the treatment group. In some cases, the operations of this step refer to, or may be performed by, a content customization apparatus as described with reference to FIGS. 1-2. For example, in some cases, the content customization apparatus provides the customized content to the treatment group as described with reference to FIG. 6.

At operation 515, the system receives outcome data for the treatment group. In some cases, the operations of this step refer to, or may be performed by, a content customization apparatus as described with reference to FIGS. 1-2. For example, in some cases, the content customization apparatus receives the outcome data for the treatment group as described with reference to FIG. 6.

At operation 520, the system obtains an estimated treatment effect for a user based on the outcome data. In some cases, the operations of this step refer to, or may be performed by, a content customization apparatus as described with reference to FIGS. 1-2. For example, in some cases, the content customization apparatus obtains the estimated treatment effect for the user as described with reference to FIG. 6.

At operation 525, the system provides the customized content to the user based on the estimated treatment effect. In some cases, the operations of this step refer to, or may be performed by, a content customization apparatus as described with reference to FIGS. 1-2. For example, in some cases, the content customization apparatus provides the customized content to the user based on the estimated treatment effect as described with reference to FIG. 6.

FIG. 6 shows an example of customizing content based on an individual treatment effect estimator according to aspects of the present disclosure. In some examples, these operations are performed by a system including a processor executing a set of codes to control functional elements of an apparatus. Additionally or alternatively, certain processes are performed using special-purpose hardware. Generally, these operations are performed according to the methods and processes described in accordance with aspects of the present disclosure. In some cases, the operations described herein are composed of various substeps, or are performed in conjunction with other operations.

Referring to FIG. 6, according to some aspects, a content customization system determines a control group of users and a treatment group of users from a set including n users, where s is a sum of a number of users included in the treatment group and a number of users included in the control group, based on d user feature vectors (e.g., covariates). According to some aspects, s is minimized when s=Θ(d log d), where Θ is a constant. According to some aspects, s is optimal up to a \sqrt{log d} factor.

In some cases, by determining the control group and the treatment group based on the user features, the content customization system minimizes a number of users for determining an estimated treatment effect for a user of the set of users. According to some aspects, the content customization system trains an individual treatment effect estimator based on the control group and the treatment group using a machine learning model. According to some aspects, the content customization system provides customized content to the user based on the individual treatment effect estimator.

At operation 605, the system identifies a user feature matrix that represents user features for each of a set of users. In some cases, the operations of this step refer to, or may be performed by, a user feature component as described with reference to FIGS. 2-4.

According to some aspects, the user feature component receives and identifies a user feature matrix X ∈ [symbol] ^{n×d} for a set of n users, where n is an integer and d is a number of user feature vectors (e.g., covariates) for the set of n users. For example, in some cases, for the set of n users, each user in the set of users is represented with an integer in [n], where [n] [symbol] {1,2, . . . , n}. In some cases, each row of the user feature matrix X comprises a user feature vector representing user features for a user in the set of users n. In some cases, X[i, :] and X[j, :] denotes an i^{th} row and a j^{th} row of the user feature matrix X, respectively, and X[:, i] and X[:, j] denotes an i^{th} column and a j^{th} column of the user feature matrix X, respectively. In some cases, each column of the user feature matrix X is a column vector.

In some cases, the user feature matrix X is row-normalized; i.e., |X[i, :]∥≤1 ∀i ∈ [n]. In some cases, an i^{th} largest singular value of X is denoted by σᵢ(X). In some cases, for any given vector p, a Euclidean norm or an [symbol] ₂-norm is denoted by ∥P∥.

According to some aspects, the user feature component generates the user feature vectors and the user feature matrix X by embedding the corresponding user features. According to some aspects, a content component as described with reference to FIGS. 2-4 receives the corresponding user features by monitoring a content channel and provides the user features to the user feature component.

According to some aspects, the user feature component performs a smoothing operation on the user feature matrix X to obtain a smoothed user feature matrix X*. For example, in some cases, the user feature component projects the user feature matrix X onto singular vectors of the user feature matrix X that correspond to high singular values (e.g., singular values that exceed a singular value threshold) to obtain the smoothed user feature matrix X*. According to some aspects, the smoothed user feature matrix X* reduces the effects of high leverage score (i.e., outlier) rows of the user feature matrix X that do not contribute significantly to the spectrum of the user feature matrix X.

For example, in some cases, given X ∈ [symbol] ^{n×d} with singular value decomposition X=UΣV^{T}, Γ* is a set of indices corresponding to singular values greater than \sqrt{γ}, i.e., Γ* [symbol] {i|σᵢ(X)≥\sqrt{λ}}, for some λ≥0. In some cases, d′ [symbol] |Γ*|. In some cases, Σ*=Σ(Γ*, Γ*) is a principal sub-matrix of Σ associated with the large singular values. Similarly, in some cases, U* ∈ [symbol] ^{n×d′}, V* ∈ [symbol] ^{d×d′} are associated column sub-matrices of U and V, respectively. Accordingly, the smoothed user feature matrix X* is computed by the user feature component as X* [symbol] U*Σ*V*^{T}. In some cases, the user feature component obtains U* by taking rows of U such that the corresponding similar values are

$$Ω ( max \{ log d , \frac{s}{d} \} ) .$$

At operation 610, the system computes a leverage score for each of the set of users based on the user feature matrix. In some cases, the operations of this step refer to, or may be performed by, a group selection component as described with reference to FIGS. 2-4.

In some cases, for a j^{th} row X[j, :] of the user feature matrix X or a j^{th} row X*[j, :] of the smoothed user feature matrix X*, the group selection component computes a leverage score [symbol] ⱼ(X) [symbol] X[j, :]^{T}(X^{T}X)⁺X[j, :] or a leverage score [symbol] ⱼ(X*)=X*[j, :]^{T}(X*^{T}X*)⁺X[j, :], respectively, where + denotes a Moore-Penrose inverse (e.g., a pseudoinverse, which is a generalization of an inverse matrix). In some cases, the leverage score [symbol] ⱼ(X) or [symbol] ⱼ(X*) for a user j is a numerical representation of an importance of a j^{th} row corresponding to the user j to the row space of the user feature matrix X or of the smoothed user feature matrix X*. For example, Σ_{j∈[n]} [symbol] ⱼ(X)=rank(X) and Σ_{j∈[n]} [symbol] ⱼ(X*)=rank(X*). In some cases, if a j^{th} row is orthogonal to other rows of the user feature matrix X or of the smoothed user feature matrix X*, the leverage score of the j^{th} row will have a maximum value of 1.

At operation 615, the system generates a treatment sampling matrix for a treatment group and a control sampling matrix for a control group based on the leverage score. In some cases, the operations of this step refer to, or may be performed by, a group selection component as described with reference to FIGS. 2-4.

According to some aspects, the group selection component generates a selection probability function based on the leverage score [symbol] ⱼ(X) or [symbol] ⱼ(X*) for each of the plurality of users. For example, in some cases, the group selection component receives each of the leverage scores from the user feature component. In some cases, the group selection component generates a selection probability function π ∈ [symbol] ⁿ, where a probability π of a row of the user feature matrix X or of the smoothed user feature matrix X* being selected is proportional to a corresponding leverage score [symbol] ⱼ(X) or [symbol] ⱼ(X*), respectively. According to some aspects, the group selection component selects π such that s=Θ(d log d), thereby optimizing and minimizing s.

According to some aspects, the group selection component selects the treatment group and the control group based on the selection probability function. In some cases, the group selection component receives the user feature matrix X or the smoothed user feature matrix X* from the user selection component. In some cases, the group selection component performs row sampling on the user feature matrix X or the smoothed user feature matrix X*, respectively, to independently include, with a probability πⱼ, a j^{th} row corresponding to a user j ∈ [n] of the user feature matrix X or of the smoothed user feature matrix X* in a control set S⁰ corresponding to the control group, or, with a probability πⱼ(1−πⱼ), the j^{th} row in a treatment set S¹ corresponding to the treatment group. According to some aspects, by sampling from the smoothed user feature matrix X*, the group selection component reduces a re-sampling of users.

In some cases, leverage scores of the smoothed user feature matrix X*, and in turn the probability π, are bounded by 1/γ, and therefore the sampling probability for S¹, π(1−π), is not far from π. In some cases, row norms of the user feature matrix X are bounded by 1, and row norms of the smoothed user feature matrix X* are also bounded. Therefore, in some cases, there can be no rows in the smoothed user feature matrix X* that are nearly orthogonal to other rows; i.e., in some cases, the smoothed user feature matrix X* does not include a row that corresponds to a very high leverage score. In some cases, a smallest singular value of the smoothed user feature matrix X* is \sqrt{γ}, and [symbol] ⱼ(X*)≤1/γ for all users j ∈ [n].

For example, in some cases, [symbol] ⱼ(X*)=X*[j,:]^{T}(X*^{T}X*)⁺X[j,:]=∥U*[j,:]∥². Furthermore, in some cases, ∥X*[j,:]∥²≤∥X[j,:]∥², and therefore ∥X*[j,:]∥²=∥Σ*U*[j,:]∥²≤1. Accordingly, as all diagonal entries of Σ* are at least \sqrt{γ}, [symbol] ⱼ(X*)=∥U*[j,:]∥²≤1/γ.

In some cases, the group selection component identifies a user in the treatment group and the control group and removes the user from the treatment group or the control group. For example, in some cases, the j^{th} row corresponding to the user j is included in both the control set S⁰ and the treatment set S¹ after sampling. In this case, the group selection component removes the j^{th} row from the control set S⁰ or the treatment set S¹. According to some aspects, as the probability π for sampling rows from the smoothed user feature matrix X* is bounded, few rows are randomly assigned to both the control set S⁰ or the treatment set S¹, and therefore an error that may be introduced by removing a row from the control set S⁰ or the treatment set S¹ is reduced.

According to some aspects, the group selection component generates the control sampling matrix W⁰ for the control group by including each row in the control set S⁰ as a row in the control sampling matrix W⁰. According to some aspects, the group selection component generates the treatment sampling matrix W¹ for the treatment group by including each row in the treatment set S¹ as a row in the treatment sampling matrix W¹.

According to some aspects, the group selection component samples users corresponding to rows of the smoothed user feature matrix X* independently, such that an i^{th} row of the smoothed user feature matrix X* is included in the sample with some probability πᵢ, and a set of sampled rows is denoted by S. In some cases, a j^{th} row of a sampling matrix W is associated with a j^{th} element in the set S (under some fixed order). If the j^{th} element in the set S is the row for a user i for some i ∈ [n], then W[j,:]=eᵢ/\sqrt{πᵢ}, where eᵢ ∈ [symbol] ⁿ denotes the i^{th} standard basis vector. In this way, WX* can comprise a subset of rows sampled in S, reweighted by an inverse square root of their sampling probabilities, which aids a machine learning model as described with reference to FIG. 2-4 in keeping expectations correct in solving a linear regression based on the smoothed user feature matrix X*.

In some cases, an error introduced by using the smoothed user feature matrix X* in place of the user feature matrix X is small, and depends on a threshold y used in the construction of the smoothed user feature matrix X*.

According to some aspects, using the singular value decomposition of X,X*:

∥X*β−Xβ∥=∥U*Σ*V*β−UΣVβ∥≤∥U*Σ*V*−UΣV∥₂·∥β∥ (1)

In some cases, ∥·∥₂ denotes a spectral norm (i.e., a largest singular value) of a matrix. In some cases, by construction, ∥U*Σ*V*−UΣV∥₂≤\sqrt{γ}, and therefore, for every β ∈ [symbol] ^{d}, ∥X*β−Xβ∥≤\sqrt{γ}·∥β∥.

At operation 620, the system trains an individual treatment effect estimator using a machine learning model based on the treatment sampling matrix, the control sampling matrix, the user feature matrix, and outcome data for the set of users. In some cases, the operations of this step refer to, or may be performed by, a machine learning model as described with reference to FIGS. 2-4.

According to some aspects, a content component as described with reference to FIGS. 2-4 receives the control sampling matrix W⁰ and the treatment sampling matrix W¹ from the group selection component. In some cases, the content component identifies each user corresponding to the treatment sampling matrix W¹ as belonging to the treatment group and provides customized content to the users belonging to the treatment group. In some cases, the customized content is stored in a database (such as the database as described with reference to FIG. 1). In some cases, the customized content is received from a third-party user (such as an organization).

The content component monitors a treatment outcome (e.g., an action taken by the user belonging to the treatment group) in response to the users receiving the customized content to obtain treatment outcome data. In some cases, the content component monitors the treatment outcome via a communication with a content channel. In some cases, the content component monitors the treatment outcome via API calls to the content channel or other similar techniques. In some cases, the content component determines a treatment outcome vector y¹ based on the treatment outcome data. In some cases, the treatment outcome vector y¹ is based on an average of the treatment outcome data for each user in the treatment group.

In some cases, the content component identifies each user corresponding to the control sampling matrix W⁰ as belonging to the control group and provides content other than the customized content to the users belonging to the control group. The content component monitors a control outcome (e.g., an action taken by the user belonging to the control group) in response to the users receiving the content to obtain control outcome data. In some cases, the content component monitors the control outcome via a communication with a content channel. In some cases, the content component determines a control outcome vector y⁰ based on the control outcome data. In some cases, the control outcome vector y⁰ is based on an average of the control outcome data for each user in the control group.

In some cases, the content component does not provide content to the users belonging to the control group, and monitors actions taken by the users of the control group independently of receiving content to obtain the control outcome data.

According to some aspects, the machine learning model receives the control sampling matrix W⁰ and the treatment sampling matrix W¹ from the group selection component. According to some aspects, the machine learning model receives the user feature matrix X or the smoothed user feature matrix X* from the user feature component. According to some aspects, the machine learning model receives the treatment outcome vector y¹ and the control outcome vector y⁰ from the content component.

According to some aspects, the machine learning model comprises a regression of a treatment outcome function (e.g., the treatment outcome vector y¹) and a control outcome function (e.g., the control outcome vector y⁰) on the user feature matrix X or on the smoothed user feature matrix X*. In some cases, the treatment outcome function and the control outcome function are linear functions of the user feature vectors (e.g., the covariates). In some cases, the treatment outcome function and the control outcome function are linear functions of the covariates under a linearity assumption. Formally, for some β⁰, β¹ ∈ [symbol] ^{d}, y¹=Xβ¹+ζ¹ and y⁰=Xβ⁰+ζ⁰, where ζ¹, ζ⁰ ∈ [symbol] ⁿ are noise vectors, with each coordinate drawn independently from the Gaussian distribution having zero mean and variance σ² (i.e., N(0, σ²)). In some cases, ζ ∈ [symbol] ⁿ is a vector such that each co-ordinate ζᵢ is drawn independently from the normal distribution. N(0, σ²). Then, with probability ≥1−1/n, ∥ζ∥≤2σ·\sqrt{n}.

In some cases, the machine learning model separately solves linear regressions for i=0, 1 according to equations (2) or (3), thereby training an individual treatment effect estimator:

\tilde{β}ⁱ= [symbol] ∥WⁱXβ−Wⁱyⁱ∥² (2)

\tilde{β}ⁱ= [symbol] ∥WⁱX*β−Wⁱyⁱ∥² (3)

In some cases, the individual treatment effect estimator is based on the treatment outcome function and the control outcome function. For example, for each user j ∈ [n], the machine learning model determines, based on \tilde{β}ⁱ, that an individual treatment effector estimator [symbol] (j) is a j^{th} entry in a vector X\tilde{β}¹−X\tilde{β}⁰ or a vector X*\tilde{β}¹−X*\tilde{β}⁰, respectively.

According to some aspects, the probability π corresponds to the control outcome vector y⁰. For example, in some cases, if a row of the smoothed user feature matrix X* is sampled with a probability of π proportional to a leverage score [symbol] ⱼ(X*), a (1±ϵ) relative error approximation is obtained for linear regression. If X ∈ [symbol] ^{n×d}, y ∈ [symbol] ⁿ, S ⊆ [n] includes each j ∈ [n] independently with probability πⱼ satisfying

$${π}_{j} \geq min \{ 1 , {ℓ}_{j} ( X ) \cdot c \cdot [ log ( rank ( X ) ) + \frac{1}{δϵ} ] )$$

for some large enough constant c. If W ∈ [symbol] ^{|S|×n} is a sampling matrix that includes a row eⱼ/\sqrt{πⱼ} if j ∈ S, where eⱼ ∈ [symbol] ⁿ is the j^{th} standard basis vector, and if \tilde{β}= [symbol] _{d}∥WXβ−Wy∥², then [symbol] [|S|]=Σⱼ₌₁ⁿπⱼ, and, with a probability ≥1−δ:

$$‖ X \tilde{β} - y ‖ \leq ( 1 + ϵ ) \cdot {min}_{β} ‖ X β - y ‖ \qquad ( 4 )$$

In some cases, if πⱼ is within a constant of a bound, then

$$𝔼 [ \vert S \vert ] = O ( d log d + \frac{d}{ϵδ} ) ,$$

which follows from a sum of leverage scores being equal to a rank; i.e., Σⱼ₌₁ⁿ [symbol] ⱼ(X)=rank(X)≤d. According to some aspects, then, the group selection component sets the probability πⱼ=min{1, [symbol] ⱼ(X*)·c₀·[log(rank(X*))+30/ϵ]} for some constant c₀≥2c, and therefore, with a probability ≥29/30, ∥X*\tilde{β}⁰−y⁰∥≤(1±ϵ)∥X*\tilde{β}⁰−y⁰∥. Furthermore, in some cases, as a j^{th} row of the smoothed user feature matrix X* is independently included in the treatment set S¹ with a probability of

$${π}_{j} ( 1 - {π}_{j} ) , if {π}_{j} \leq 1 / 2 , then {π}_{j} ( 1 - {π}_{j} ) \geq \frac{{π}_{j}}{2} ,$$

and equation (4) holds.

According to some aspects, if γ=4c₀max{log(rank(X*)),30/ϵ} and πⱼ=min{1, [symbol] ⱼ(X*)·c₀·[log(rank(X*))+30/ϵ]}, πⱼ≤1/2 for every j ∈ [n]. For example:

$${π}_{j} \leq {ℓ}_{j} ( {X}^{*} ) \cdot {c}_{0} \cdot [ log ( rank ( {X}^{*} ) ) + \frac{30}{ϵ} ] \leq \frac{1}{γ} \cdot {c}_{0} \cdot [ log ( rank ( {X}^{*} ) ) + 30 / ϵ ] \leq \frac{{c}_{0} \cdot [ log ( rank ( {X}^{*} ) ) + 30 / ϵ ]}{4 {c}_{0} max \{ log ( rank ( {X}^{*} ) ) , 30 / ϵ \}} \leq \frac{1}{2} . \qquad ( 5 )$$

According to some aspects, based on equation (4) and πⱼ≤1/2 for every j ∈ [n], if γ=4c₀ max{log(rank(X*)), 30/ϵ} and πⱼ=min{1, [symbol] ⱼ(X*)·c₀·[log(rank(X*))+30/ϵ]} for some sufficiently large constant c₀, then equation (3) satisfies, for i=0, 1 and with a probability of at least 14/15:

∥X*\tilde{β}ⁱ−yⁱ∥≤(1±ϵ)·∥X*βⁱ−yⁱ∥ for every i=0, 1 (6)

Furthermore, in some cases:

[symbol]

[|S⁰ ∪ S¹|]≤2Σⱼ₌₁ⁿπⱼ=0(d log d+d/ϵ) (7)

For example, if ∥X*\tilde{β}ⁱ−yⁱ∥≤(1±ϵ)·∥X*βⁱ−yⁱ∥ for every i=0, 1, using union bound, a total failure probability is

$$\leq \frac{1}{30} + \frac{1}{30} \leq \frac{1}{15} .$$

Then, given equation (4), and using Σ_{j∈[n]} [symbol] ⱼ(X*)=rank(X*) and rank(X*)≤d:

$$𝔼 [ \vert {S}^{0} ⋃ {S}^{1} \vert ] \leq 2 {\sum }_{j = 1}^{n} {π}_{j} \leq 2 {\sum }_{j ∈ [ n ]} {ℓ}_{j} ( {X}^{*} ) \cdot {c}_{0} \cdot [ log ( rank ( {X}^{*} ) ) + \frac{30}{ϵ} ] \leq 2 {c}_{0} d \cdot [ log d + 30 ϵ ] = O ( d log d + d / ϵ ) \qquad ( 8 )$$

According to some aspects, based on equation (4) and πⱼ≤1/2 for every j ∈ [n], if γ=4c₀max{log(rank(X*)), 30/ϵ} and πⱼ=min{1, [symbol] ⱼ(X*)·c₀·[log(rank(X*))+30/ϵ]} for some sufficiently large constant c₀, then equation (3) satisfies, for i=0, 1 and with a probability of at least 14/15:

∥X*\tilde{β}ⁱ−yⁱ∥≤(1±ϵ)·(\sqrt{γ}∥βⁱ∥+∥ζⁱ∥) for every i=0,1 (9)

For example, if ∥X*\tilde{β}ⁱ−yⁱ∥≤(1±ϵ)·∥X*βⁱ−yⁱ∥, then ∥X*\tilde{β}ⁱ−yⁱ∥≤(1±ϵ)·∥X*βⁱ−Xβⁱ∥+∥Xβⁱ−yⁱ∥ using triangle inequality, and therefore ∥X*\tilde{β}ⁱ−yⁱ∥≤(1±ϵ)·(\sqrt{γ}∥βⁱ∥+∥ζⁱ∥), as for every β ∈ [symbol] ^{d}, ∥X*β−Xβ∥≤\sqrt{γ}·∥β∥.

According to some aspects, a root mean squared error for the individual treatment effect estimators [symbol] (j) for all j ∈ [n] is given by:

$$RMSE = \frac{}{\sqrt{n}} ‖ ( {X}^{*} {\tilde{β}}^{1} - {X}^{*} {\tilde{β}}^{0} ) - ( {y}^{1} - {y}^{0} ) ‖ \qquad ( 10 )$$

Therefore, by setting ϵ=120c₀d log d in equation (9), if s≥120c₀d log d, there is a randomized algorithm that selects a subset S ⊆ [n] of a population with [symbol] [|S|]≤s and with probability 9/10 returns individual treatment effect estimators [symbol] (j) for all j ∈ [n] with error:

$$RMSE = O ( \sqrt{\frac{1}{n} max \{ \frac{s}{d} , log d \}} \cdot ( ‖ {β}^{1} ‖ + ‖ {β}^{0} ‖ ) + σ ) \qquad ( 11 )$$

For example, if

$$ϵ = \frac{120 {c}_{0} d log d}{s}$$

and γ=4c₀max{log d, 30/ϵ}, based on equation (9) and using triangle inequality:

∥(X*\tilde{β}¹−X*\tilde{β}⁰)−(y¹−y⁰)∥₂≤∥X*\tilde{β}⁰−y⁰∥₂≤(1±ϵ)·(\sqrt{γ}∥β¹∥+\sqrt{γ}∥β⁰∥+∥ζ⁰∥+∥ζ¹∥) (12)

Then, based on equation (12):

$$RMSE = {( \frac{1}{n} {‖ ( {X}^{*} {\tilde{β}}^{1} - {X}^{*} {\tilde{β}}^{0} ) - ( {y}^{1} - {y}^{0} ) ‖}^{2} )}^{1 / 2} \leq \frac{1}{\sqrt{n}} [ 2 \sqrt{γ} \cdot ( ‖ {β}^{1} ‖ + ‖ {β}^{0} ‖ ) + 2 \cdot ( ‖ {ζ}^{0} ‖ + ‖ {ζ}^{1} ‖ ) ] \leq \frac{1}{\sqrt{n}} [ 2 \sqrt{γ} \cdot ( ‖ {β}^{1} ‖ + ‖ {β}^{0} ‖ ) + 8 σ \sqrt{n} ] \leq 2 \sqrt{\frac{4 {c}_{0}}{n}} max \{ log d , \frac{s}{{c}_{0} d} \} \cdot ( ‖ {β}^{1} ‖ + ‖ {β}^{0} ‖ ) + 8 σ \qquad ( 13 )$$

Using union bound, a probability of failure is upper bounded by

$$\frac{1}{15} + \frac{2}{n} \leq \frac{1}{10}$$

for large n. Based on equations (6) and (7):

$$𝔼 [ \vert {S}^{0} ⋃ {S}^{1} \vert ] \leq 2 {\sum }_{j = 1}^{n} {π}_{j} \leq 2 {\sum }_{j ∈ [ n ]} {ℓ}_{j} ( {X}^{*} ) \cdot {c}_{0} \cdot [ log ( rank ( {X}^{*} ) ) + \frac{30}{ϵ} ] \leq 2 {c}_{0} d \cdot [ log d + 30 ϵ ] \leq 4 {c}_{0} d max \{ log d , \frac{s}{4 {c}_{0} d log d} \} = max \{ 4 {c}_{0} d log d , s \} \leq s \qquad ( 14 )$$

Therefore, equation (11) results.

Accordingly, in some cases, a root mean squared error obtained for individual treatment effect estimators [symbol] (j) for all j ∈ [n] is minimized when s=Θ(d log d) and is given by:

$$RMSE = O ( \sqrt{\frac{log d}{d}} \cdot ( ‖ {β}^{1} ‖ + ‖ {β}^{0} ‖ ) + σ ) \qquad ( 15 )$$

At operation 625, the system provides customized content for a user based on the individual treatment effect estimator. In some cases, the operations of this step refer to, or may be performed by, a content component as described with reference to FIGS. 2-4.

For example, in some cases, the content component receives the individual treatment effect estimator [symbol] (j) for a j^{th} user from the machine learning model. In some cases, the content component evaluates the individual treatment effect estimator to compute the estimated treatment effect for the user. In some cases, the content component determines to provide the customized content to the user based on the estimated treatment effect. For example, in some cases, the content component determines that the estimated treatment effect exceeds a treatment effect threshold. In some cases, in response to the determination, the content component provides the customized content to the user.

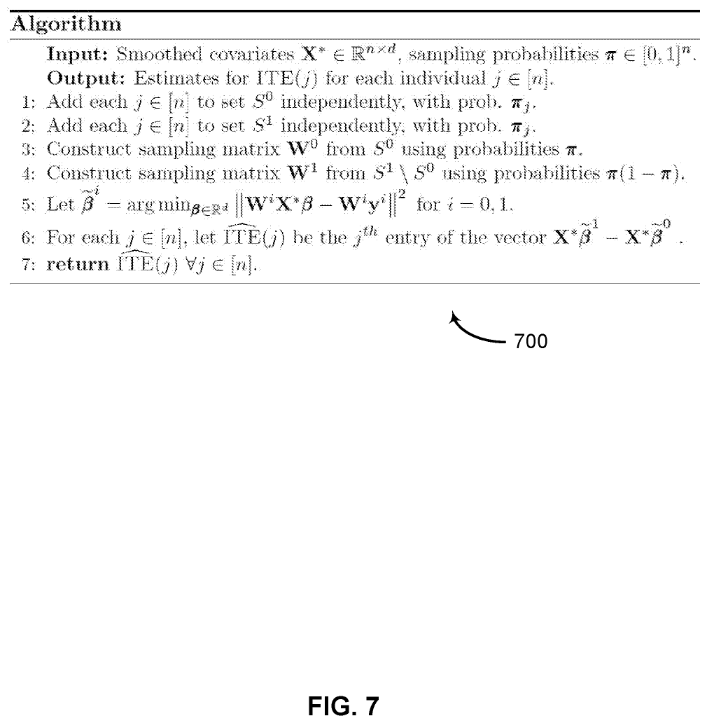

FIG. 7 shows an example of an algorithm for determining an individual treatment effect estimator according to aspects of the present disclosure. Referring to FIG. 7, algorithm 700 is an example of a process for determining an individual treatment effect estimator [symbol] (j) or a j^{th} user based on a smoothed user feature matrix X* as described with reference to FIG. 6.

### Customizing Content Based on an Average Treatment Effect Estimator

A method for content customization is described with reference to FIGS. 8-10. One or more aspects of the method include identifying a plurality of feature vectors that represent user features for a plurality of users, respectively; generating a treatment group and a control group from the plurality of users by recursively partitioning the plurality of users based on the plurality of feature vectors; training an average treatment effect estimator using a machine learning model based on outcome data for the treatment group and the control group; and providing customized content for a user based on the average treatment effect estimator.

In some cases, the partitioning is based on a Gram-Schmidt-Walk algorithm. Some examples of the method further include identifying pairs of similar users among the plurality of users. Some examples further include selecting a user from each of the pairs, wherein the partitioning is based on the selected user.

Some examples of the method further include identifying a size for the treatment group. Some examples further include selecting a number of iterations based on the size, wherein the partitioning is based on the number of iterations. In some aspects, the treatment group comprises a coreset of the plurality of users.

Some examples of the method further include providing the customized content to the treatment group. Some examples further include monitoring an outcome after providing the customized content, wherein the outcome data is obtained based on the monitoring. Some examples of the method further include computing an estimated treatment effect for the user based on the average treatment effect estimator. Some examples further include determining to provide the customized content to the user based on the estimated treatment effect.

FIG. 8 shows an example of customizing content based on an average treatment effect estimator according to aspects of the present disclosure. In some examples, these operations are performed by a system including a processor executing a set of codes to control functional elements of an apparatus. Additionally or alternatively, certain processes are performed using special-purpose hardware. Generally, these operations are performed according to the methods and processes described in accordance with aspects of the present disclosure. In some cases, the operations described herein are composed of various substeps, or are performed in conjunction with other operations.

Referring to FIG. 8, according to some aspects, the system recursively partitions a set of users based on a set of user features into a treatment group and a control group until a minimal number of users s are included in the treatment group or the control group. In some cases, based on the recursive partitioning, the system uses a machine learning model to train an average treatment effect estimator for the set of users. In some cases, the recursive portioning is based on user feature vector (e.g., covariate) balancing, where pairs of similar users are assigned to the treatment group and to the control group to ensure that an observed outcome is attributed to provided content alone. In some cases, the system uses the average treatment effect estimator to provide customized content to a user in the set of users.

At operation 805, the system identifies a set of feature vectors that represent user features for a set of users, respectively. In some cases, the operations of this step refer to, or may be performed by, a user feature component as described with reference to FIGS. 2-4. For example, in some cases, the user feature component receives a user feature matrix X ∈ [symbol] ^{n×d} including the set of d user feature vectors that represent user features for a set of n users, respectively, where n and d are integers.

At operation 810, the system generates a treatment group and a control group from the set of users by recursively partitioning the set of users based on the set of feature vectors. In some cases, the operations of this step refer to, or may be performed by, a group selection component as described with reference to FIGS. 2-4.

According to some aspects, the recursive partitioning of the set of users is based on a Gram-Schmidt-Walk algorithm. In some cases, a Gram-Schmidt-Walk algorithm uses Gram-Schmidt orthogonalization of user feature vectors to attempt to balance the user feature vectors (e.g., the covariates). For example, according to some aspects, the group selection component receives user feature matrix X, identifies similar users among the set of users corresponding to user feature matrix X based on the set of user feature vectors, and randomly selects a user from each of the pairs for inclusion in a partition. In some cases, the partitioning is based on the selected user. In some cases, the Gram-Schmidt-Walk algorithm produces a random partition of the set of users with a good balance in every dimension, such that the users selected for the treatment group and users selected for the control group correspond to similar user features.

For example, according to some aspects, in each recursive call, available users Zₜ of the set of users are partitioned by the group selection components into treatment and control groups, respectively denoted by Zₜ⁺, Zₜ⁻, using the Gram-Schmidt-Walk algorithm. Next, the group selection component identifies a smaller of the two subsets (for example, Zₜ⁺), and recurses on the smaller of the two subsets Zₜ⁺. In some cases, the group selection component stops the recursive partitioning stops after k recursive calls when there are s individuals to experiment on, where |Zₖ⁺ ∪ Zₖ⁻|≤s.

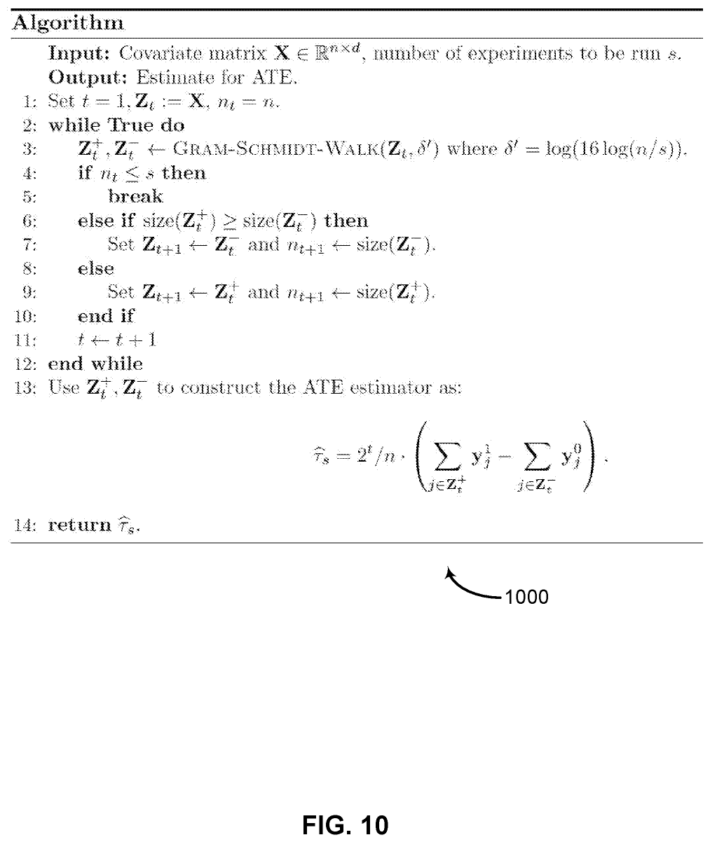

According to some aspects, the group selection component identifies a size for the treatment group and selects a number of iterations based on the size, where the partitioning is based on the number of iterations. In some cases, referring to FIG. 10, the group selection component initially sets t=1, Zₜ:=X, and nₜ=n. In some cases, the group selection component performs recursive portioning using the Gram-Schmidt-Walk algorithm. In some cases, if nₜ>s and a number of users in Zₜ⁺ is greater than or equal to a number of users in Zₜ⁻, the group selection component sets Zₜ₊₁ to Zₜ⁻ and sets nₜ₊₁ to a number of users in Zₜ⁻. In some cases, if nₜ>s and a number of users in Zₜ⁺ is less than a number of users in Zₜ⁻, the group selection component sets Zₜ₊₁ to Zₜ⁺ and sets nₜ₊₁ to a number of users in Zₜ⁺. In some cases, the group selection component stops recursively portioning if t←t+1.

According to some aspects, the treatment group Zₜ⁺ comprises a coreset of the set of users. A coreset is a small set of points that approximates the shape of a larger point set, such that applying a heuristic to the coreset and the larger point set results in approximately equal numbers. Accordingly, a model fitting the coreset will also provide a good fit for the larger point set.

At operation 815, the system trains an average treatment effect estimator using a machine learning model based on outcome data for the treatment group and the control group. In some cases, the operations of this step refer to, or may be performed by, a machine learning model as described with reference to FIGS. 2-4.

According to some aspects, a content component as described with reference to FIGS. 2-4 receives the treatment group Zₜ⁺ and the control group Zₜ⁻ from the group selection component. In some cases, the content component identifies each user included in the treatment the treatment group Zₜ⁺ and provides customized content to the users included in the treatment group the treatment group Zₜ⁺. The content component monitors a treatment outcome (e.g., an action taken by the user belonging to the treatment group Zₜ⁺) in response to the users receiving the customized content to obtain treatment outcome data. In some cases, the content component monitors the treatment outcome via a communication with a content channel. In some cases, the content component determines a treatment outcome vector y¹ based on the treatment outcome data. In some cases, the treatment outcome vector y¹ is based on an average of the treatment outcome data for the treatment group Zₜ⁺.

In some cases, the content component identifies each user included in the control group Zₜ⁻ and provides content other than the customized content to the users belonging to the control group Zₜ⁻. The content component monitors a control outcome (e.g., an action taken by the user belonging to the control group Zₜ⁻) in response to the users receiving the content to obtain control outcome data. In some cases, the content component monitors the control outcome via a communication with a content channel. In some cases, the content component determines a control outcome vector y⁰ based on the control outcome data. In some cases, the control outcome vector y⁰ is based on an average of the control outcome data for the control group Zₜ⁻.

In some cases, the content component does not provide content to the users belonging to the control group the control group Zₜ⁻, and monitors actions taken by the users of the control group Zₜ⁻ independently of receiving content to obtain the control outcome data.

According to some aspects, a machine learning model as described with reference to FIGS. 2-4 receives the user feature matrix X from the user feature component, the treatment group Zₜ⁺ and the control group Zₜ⁻ from the group selection component, and the treatment outcome vector y¹ and the control outcome vector y⁰ from the content component. According to some aspects, the machine learning model trains an average treatment effect estimator \hat{τ}ₛ by scaling the treatment and control contributions due to Zₜ⁺ and Zₜ⁻, where t is the total number of iterations of covariate-balancing:

$${\hat{τ}}_{s} = \frac{{2}^{t}}{n} ( {\sum }_{j ∈ {Z}_{t}^{+}} {y}_{j}^{1} - {\sum }_{j ∈ {Z}_{t}^{-}} {y}_{j}^{0} ) \qquad ( 16 )$$

At operation 820, the system provides customized content for a user based on the average treatment effect estimator. In some cases, the operations of this step refer to, or may be performed by, a content component as described with reference to FIGS. 2-4.

For example, in some cases, the content component receives the average treatment effect estimator for the user from the machine learning model. In some cases, the content component evaluates the average treatment effect estimator to compute the estimated treatment effect for the user. In some cases, the content component determines to provide the customized content to the user based on the estimated treatment effect. For example, in some cases, the content component determines that the estimated treatment effect exceeds a treatment effect threshold. In some cases, in response to the determination, the content component provides the customized content to the user.

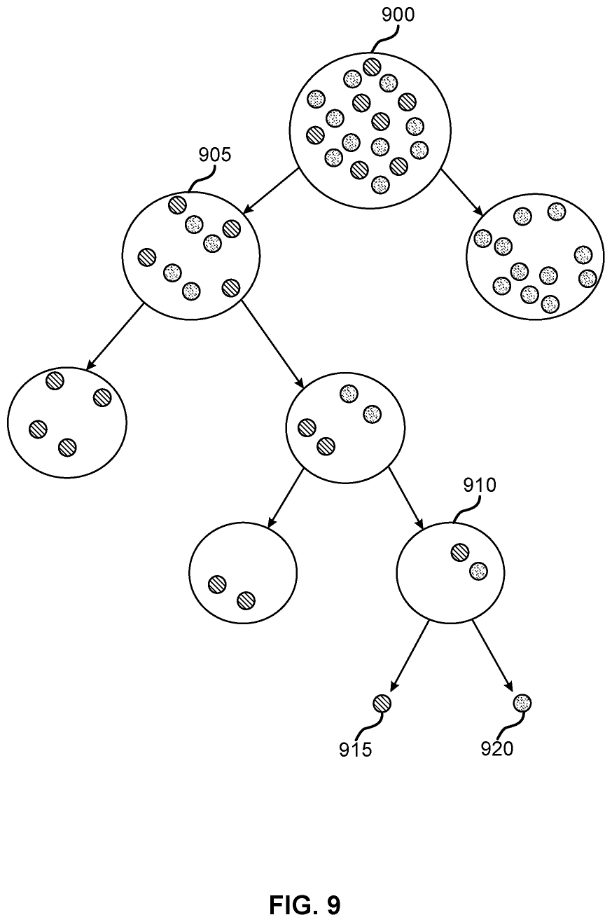

FIG. 9 shows an example of partitioning a set of users according to aspects of the present disclosure. The example shown includes set of users 900, first partition 905, n^{th} partition 910, treatment group user 915, and control group user 920. Referring to FIG. 9, a group selection component as described with reference to FIGS. 2-4 recursively partitions set of users 900 as described with reference to FIG. 8 to obtain first partition 905 through n^{th} partition 910, where n^{th} partition 910 is used to identify a treatment group and a control group. The treatment group includes treatment group user 915, and the control group includes control group user 920.

FIG. 10 shows an example of an algorithm for determining an average treatment effect estimator according to aspects of the present disclosure. Referring to FIG. 10, algorithm 1000 is an example of a process for determining an average treatment effect estimator \hat{τ}ₛ for a set of users based on a user feature matrix X as described with reference to FIG. 8.

According to some aspects, the following bounded error is obtained for the average treatment effect estimator \hat{τ}ₛ, with probability at least 2/3:

$$\vert {\hat{τ}}_{s} - τ \vert = O ( \sqrt{log log ( n / s )} \cdot ( \frac{σ}{\sqrt{s}} + \frac{‖ {β}^{1} ‖ + ‖ {β}^{0} ‖}{s} ) ) \qquad ( 17 )$$

For example, according to some aspects, for all Δ>0, with a probability of at least

$$1 - 2 exp ( - \frac{{Δ}^{2} n}{8 l} ) , where L = \frac{2}{n} {min}_{β ∈ {ℝ}^{d}} ( {‖ \frac{{y}^{1} + {y}^{0}}{2} - X β ‖}^{2} + {‖ β ‖}^{2} ) ,$$

algorithm 1000 satisfies

$$\vert \hat{τ} - τ \vert \leq Δ , where \hat{τ} = \frac{2}{n} ( {Σ}_{i ∈ {S}^{+}} {y}_{i}^{1} - {Σ}_{i ∈ {S}^{-}} {y}_{i}^{0} )$$

is a comparative average treatment effect estimator for population subsets S⁺, S⁻, where S⁺ ⊆ [n] is a subset assigned to a treatment group with probability 0.5 and S⁻=[n]\S⁺ is a remainder of the population assigned (e.g., a control group), and τ is an actual average treatment effect. According to some aspects, by obtaining Σ_{j∈Z}_{ₜ}_{⁺}yⱼ¹ and Σ_{j∈Z}_{ₜ}_{^{−}}yⱼ⁰ as good estimates for Σ_{i∈S}_{⁺}yᵢ¹ and Σ_{i∈S}_{^{−}}yᵢ⁰, respectively, the average treatment effect estimator \hat{τ}ₛ is obtained according to algorithm 1000 as a good estimate for the actual average treatment effect τ.

For example, in some cases, if a set of users [n] is partitioned into two disjoint groups S⁺, S⁻, under a linearity assumption, with probability

$$1 - \frac{1}{3} log ( n / s ) ,$$

for both the treatment group S⁺ and the control group S⁻, the following holds:

|Σ_{j∈S}_{⁺}2yⱼⁱ−Σ_{j∈[n]}yⱼⁱ|≤4{square root over (log(16 log(n/s)))}·(2σ\sqrt{n}+∥βⁱ∥) for i=0,1 (18)

For example, in some cases, a covariate matrix X but not treatment and control values y¹, y⁰ are used for constructing the partitions S⁺, S⁻. In a setting where yᵢ¹=y₀¹ for all i ∈ [n], the comparative average treatment effect estimator \hat{τ} and the actual average treatment effect τ=0 satisfy:

$$\hat{τ} - τ = \frac{2}{n} ( {\sum }_{i ∈ {S}^{+}} {y}_{i}^{1} - {\sum }_{i ∈ {S}^{-}} {y}_{i}^{0} ) = \frac{2}{n} ( {\sum }_{i ∈ {S}^{+}} {y}_{i}^{1} - {\sum }_{i ∈ {S}^{-}} {y}_{i}^{1} ) \qquad ( 19 )$$

In some cases, as algorithm 1000 satisfies |\hat{τ}−τ|≤Δ:

$$L = \frac{2}{n} {min}_{β ∈ {ℝ}^{d}} ( {‖ {y}^{1} - X β ‖}^{2} + {‖ β ‖}^{2} ) \leq \frac{2}{n} ( {‖ {y}^{1} - X β ‖}^{2} + {‖ β ‖}^{2} ) \leq \frac{2}{n} ( {‖ {y}^{1} - X {β}^{1} ‖}^{2} + {‖ {β}^{1} ‖}^{2} ) = \frac{2}{n} ( {‖ {ζ}^{1} ‖}^{2} + {‖ {β}^{1} ‖}^{2} ) \qquad ( 20 )$$

In some cases, as algorithm 1000 satisfies |\hat{≢}−τ|≤Δ, then with probability at least 1−2/log(16 log(n/s)):

$$\hat{τ} - τ = \vert \frac{2}{n} ( {\sum }_{i ∈ {S}^{+}} {y}_{i}^{1} - {\sum }_{i ∈ {S}^{-}} {y}_{i}^{1} ) \vert \leq \sqrt{\frac{16 log ( 16 log ( n / s ) )}{{n}^{2}} ( {‖ {ζ}^{1} ‖}^{2} + {‖ {β}^{1} ‖}^{2} )} \leq \frac{4 \sqrt{log ( 16 log ( n / s ) )}}{n} \cdot ‖ {ζ}^{1} ‖ + ‖ {β}^{1} ‖ \qquad ( 21 )$$

Then, if ∥ζ∥≤2σ·\sqrt{n} with probability ≥1−1/n:

$${Δ}^{1} = 4 \sqrt{16 ( 16 log ( n / s ) )} \cdot ‖ {ζ}^{1} ‖ + ‖ {β}^{1} ‖ \leq 4 \sqrt{log ( 16 log ( n / s ) )} \cdot 2 σ \sqrt{n} + ‖ {β}^{1} ‖ ) \qquad ( 22 )$$

$${\sum }_{i ∈ {S}^{-}} {y}_{i}^{1} \geq {\sum }_{i ∈ {S}^{+}} {y}_{i}^{1} - {Δ}^{1} \\ {\sum }_{i ∈ {S}^{-}} {y}_{i}^{1} + {\sum }_{i ∈ {S}^{+}} {y}_{i}^{1} \geq {\sum }_{i ∈ {S}^{+}} {y}_{i}^{1} + {\sum }_{i ∈ {S}^{+}} {y}_{i}^{1} - {Δ}^{1} \\ {\sum }_{i ∈ [ n ]} {y}_{i}^{1} \geq {\sum }_{i ∈ {S}^{+}} 2 {y}_{i}^{1} - {Δ}^{1} \\ ⇒ 2 {\sum }_{i ∈ {S}^{+}} {y}_{i}^{1} - {\sum }_{i ∈ [ n ]} {y}_{i}^{1} \leq {Δ}^{1} \qquad ( 23 )$$

Similarly, Σ_{i∈[n]}yᵢ¹−2Σ_{i∈S}_{⁺}yᵢ¹+yᵢ¹≤Δ¹. In some cases, using union bound, the inequality holds with probability at least

$$q - \frac{2}{16 log ( n / s )} - \frac{1}{n} \geq 1 - \frac{1}{6 log ( n / s )} .$$

A similar bound for y⁰ can be obtained using the set S⁻.

Therefore, in a case where algorithm 1000 terminates after k≤[log(n/s)] recursive calls to the Gram-Schmidt-Walk algorithm, the average treatment effect estimator \hat{τ}ₛ is scaled using 2^{k}. If S⁺ and S⁻ denote Zₜ⁺ and Zₜ⁻, respectively, a scaled contribution of treatment values, i.e., Σ_{j∈S}_{⁺}2^{k}·yⱼ¹, is close to the contribution on the entire population, i.e., Σ_{j∈[n]}yⱼ¹. Accordingly, as this holds for both the control group and the treatment group, in some cases, the average treatment effect estimator \hat{τ}ₛ has low error.

For example:

$$\begin{matrix}{\hat{τ}}_{s} - τ = \frac{{2}^{k}}{n} ( {\sum }_{j ∈ {S}^{+}} {y}_{j}^{1} - {\sum }_{j ∈ {S}^{-}} {y}_{j}^{0} ) - \frac{1}{n} ( {\sum }_{i ∈ [ n ]} {y}_{i}^{1} - {\sum }_{i ∈ [ n ]} {y}_{i}^{0} ) & ( 24 ) \\ n \vert {\hat{τ}}_{s} - τ \vert \leq \vert {\sum }_{j ∈ {S}^{+}} {2}^{k} \cdot {y}_{j}^{1} - {\sum }_{i ∈ [ n ]} {y}_{i}^{1} \vert + \vert {\sum }_{j ∈ {S}^{-}} {2}^{k} \cdot {y}_{j}^{0} - {\sum }_{i ∈ [ n ]} {y}_{i}^{0} \vert & ( 25 )\end{matrix}$$

Then, where Σ_{j∈S}_{⁺}_{∪S}_{^{−}}yⱼ¹ are added to and subtracted from the first term in equation (24):

$$\vert {\sum }_{j ∈ {S}^{+}} {2}^{k} \cdot {y}_{j}^{1} - {\sum }_{i ∈ [ n ]} {y}_{i}^{1} \vert \leq \vert {2}^{k - 1} ( {\sum }_{j ∈ {S}^{+}} 2 \cdot {y}_{j}^{1} - {\sum }_{i ∈ {Z}_{k}} {y}_{j}^{1} ) \vert + \vert {\sum }_{j ∈ {Z}_{k}} {2}^{k - 1} \cdot {y}_{j}^{1} - {\sum }_{i ∈ [ n ]} {y}_{i}^{1} \vert \leq \vert {2}^{k - 1} \cdot 4 \sqrt{log ( 16 log ( n / s ) )} ( 2 σ \sqrt{\vert {Z}_{k} \vert} + ‖ {β}^{1} ‖ ) \vert + \vert {\sum }_{j ∈ {Z}_{k}} {2}^{k - 1} \cdot {y}_{j}^{1} - {\sum }_{i ∈ [ n ]} {y}_{i}^{1} \vert \qquad ( 26 )$$

The last step of equation (26) follows from equation (18). Then, repeating equation (26) k times provides:

$$\begin{matrix}\vert {\sum }_{j ∈ {Z}_{k}} {2}^{k - 1} \cdot {y}_{j}^{1} - {\sum }_{i ∈ [ n ]} {y}_{i}^{1} \vert \leq 4 \sqrt{log ( 16 log ( n / s ) )} \cdot {2}^{k} \cdot [ ( \frac{\sqrt{\vert {Z}_{k} \vert}}{1} \frac{\sqrt{\vert {Z}_{k - 1} \vert}}{2} + ⋯ + \frac{\sqrt{\vert {Z}_{1} \vert}}{{2}^{k}} ) σ + ( \frac{1}{2} + ⋯ + \frac{1}{{2}^{k}} ) ‖ {β}^{1} ‖ ] \leq 4 \sqrt{log ( 16 log ( n / s ) )} \cdot \frac{n}{s} \cdot [ ( \frac{\sqrt{s}}{1} + \frac{\sqrt{2 s}}{2} + ⋯ + \frac{s \sqrt{n}}{n} ) σ + ‖ {β}^{1} ‖ ] & ( 27 ) \\ \vert \frac{1}{n} ( {\sum }_{j ∈ {S}^{+}} {2}^{k} \cdot {y}_{j}^{1} - {\sum }_{i ∈ [ n ]} {y}_{i}^{1} ) \vert \leq 4 \sqrt{log ( 16 log ( n / s ) )} \cdot [ ( \frac{1}{\sqrt{s}} + \frac{1}{2 \sqrt{s}} + \frac{1}{4 \sqrt{s}} + ⋯ + \frac{1}{\sqrt{n}} ) σ + \frac{‖ {β}^{1} ‖}{s} ] \leq 4 \sqrt{log ( 16 log ( n / s ) )} \cdot ( \frac{4 σ}{\sqrt{s}} + \frac{‖ {β}^{1} ‖}{s} ) & ( 28 )\end{matrix}$$

$$Similarly :$$

$$\vert \frac{1}{n} ( {\sum }_{j ∈ {S}^{-}} {2}^{k} \cdot {y}_{j}^{0} - {\sum }_{i ∈ [ n ]} {y}_{i}^{0} ) \vert \leq 4 \sqrt{log ( 16 log ( n / s ) )} \cdot ( \frac{4 σ}{\sqrt{s}} + \frac{‖ {β}^{0} ‖}{s} ) \qquad ( 29 )$$

Accordingly, using union bound, in some cases the total failure probability is upper bounded by

$$\frac{1}{3 log ( n / s )} \cdot log ( n / s ) \leq \frac{1}{3} ,$$

and therefore, the bounded error is obtained. According to some aspects, then, a better dependence is obtained for an average treatment effect estimator obtains as compared to sampling rows uniformly at random and using outcome values corresponding to the randomly sampled rows to estimate a population mean of a treatment group and a control group.

The description and drawings described herein represent example configurations and do not represent all the implementations within the scope of the claims. For example, the operations and steps may be rearranged, combined, or otherwise modified. Also, structures and devices may be represented in the form of block diagrams to represent the relationship between components and avoid obscuring the described concepts. Similar components or features may have the same name but may have different reference numbers corresponding to different figures.

Some modifications to the disclosure may be readily apparent to those skilled in the art, and the principles defined herein may be applied to other variations without departing from the scope of the disclosure. Thus, the disclosure is not limited to the examples and designs described herein, but is to be accorded the broadest scope consistent with the principles and novel features disclosed herein.

The described methods may be implemented or performed by devices that include a general-purpose processor, a digital signal processor (DSP), an application specific integrated circuit (ASIC), a field programmable gate array (FPGA) or other programmable logic device, discrete gate or transistor logic, discrete hardware components, or any combination thereof. A general-purpose processor may be a microprocessor, a conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration). Thus, the functions described herein may be implemented in hardware or software and may be executed by a processor, firmware, or any combination thereof. If implemented in software executed by a processor, the functions may be stored in the form of instructions or code on a computer-readable medium.

Computer-readable media includes both non-transitory computer storage media and communication media including any medium that facilitates transfer of code or data. A non-transitory storage medium may be any available medium that can be accessed by a computer. For example, non-transitory computer-readable media can comprise random access memory (RAM), read-only memory (ROM), electrically erasable programmable read-only memory (EEPROM), compact disk (CD) or other optical disk storage, magnetic disk storage, or any other non-transitory medium for carrying or storing data or code.

Also, connecting components may be properly termed computer-readable media. For example, if code or data is transmitted from a website, server, or other remote source using a coaxial cable, fiber optic cable, twisted pair, digital subscriber line (DSL), or wireless technology such as infrared, radio, or microwave signals, then the coaxial cable, fiber optic cable, twisted pair, DSL, or wireless technology are included in the definition of medium. Combinations of media are also included within the scope of computer-readable media.

In this disclosure and the following claims, the word “or” indicates an inclusive list such that, for example, the list of X, Y, or Z means X or Y or Z or XY or XZ or YZ or XYZ. Also the phrase “based on” is not used to represent a closed set of conditions. For example, a step that is described as “based on condition A” may be based on both condition A and condition B. In other words, the phrase “based on” shall be construed to mean “based at least in part on.” Also, the words “a” or “an” indicate “at least one.”

What is claimed is:

1. A method for content customization, comprising:
identifying, by a user feature component, a user feature matrix that represents user features for each of a plurality of users;
computing, by a group selection component, a leverage score for each of the plurality of users based on the user feature matrix;
generating, by the group selection component, a treatment sampling matrix for a treatment group and a control sampling matrix for a control group based on the leverage score;
training, by a machine learning model, an individual treatment effect estimator based on the treatment sampling matrix, the control sampling matrix, the user feature matrix, and outcome data for the plurality of users; and
providing, by a content component, customized content for a user based on the individual treatment effect estimator.

2. The method of claim 1, further comprising:
performing, by the user feature component, a smoothing operation on the user feature matrix to obtain a smoothed user feature matrix, wherein the treatment sampling matrix is based on the smoothed user feature matrix.

3. The method of claim 1, further comprising:
generating, by the group selection component, a selection probability function based on the leverage score for each of the plurality of users; and
selecting, by the group selection component, the treatment group and the control group based on the selection probability function.

4. The method of claim 3, further comprising:
identifying, by the group selection component, a user in the treatment group and the control group; and
removing, by the group selection component, the user from the treatment group or the control group.

5. The method of claim 3, further comprising:
providing, by the content component, the customized content to the treatment group; and
monitoring, by the content component, an outcome after providing the customized content, wherein the outcome data is obtained based on the monitoring.

6. The method of claim 1, wherein:
the machine learning model comprises a regression on a treatment outcome function and a control outcome function, wherein the individual treatment effect estimator is based on the treatment outcome function and the control outcome function.

7. The method of claim 1, further comprising:
computing, by the content component, an estimated treatment effect for the user based on the individual treatment effect estimator; and
determining, by the content component, to provide the customized content to the user based on the estimated treatment effect.

8. A method for content customization, comprising:
identifying, by a user feature component, a plurality of feature vectors that represent user features for a plurality of users, respectively;
generating, by a group selection component, a treatment group and a control group from the plurality of users by recursively partitioning the plurality of users based on the plurality of feature vectors;
training, by a machine learning model, an average treatment effect estimator based on outcome data for the treatment group and the control group; and
providing, by a content component, customized content for a user based on the average treatment effect estimator.

9. The method of claim 8, further comprising:
the partitioning is based on a Gram-Schmidt-Walk algorithm.

10. The method of claim 8, further comprising:
identifying, by the group selection component, pairs of similar users among the plurality of users; and
selecting, by the group selection component, a user from each of the pairs, wherein the partitioning is based on the selected user.

11. The method of claim 8, further comprising:
identifying, by the group selection component, a size for the treatment group; and
selecting, by the group selection component, a number of iterations based on the size, wherein the partitioning is based on the number of iterations.

12. The method of claim 8, wherein:
the treatment group comprises a coreset of the plurality of users.

13. The method of claim 8, further comprising:
providing, by the content component, the customized content to the treatment group; and
monitoring, by the content component, an outcome after providing the customized content, wherein the outcome data is obtained based on the monitoring.

14. The method of claim 8, further comprising:
computing, by the content component, an estimated treatment effect for the user based on the average treatment effect estimator; and
determining, by the content component, to provide the customized content to the user based on the estimated treatment effect.

15. An apparatus for content customization, comprising:
a processor;
a memory storing instructions executable by the processor;
a user feature component configured to generate user feature vectors representing user features for a plurality of users, respectively;
a group selection component configured to select a treatment group and a control group based on the user feature vectors;
a machine learning model configured to train a treatment effect estimator based on the user feature vectors and outcome data for the treatment group and the control group; and
a content component configured to provide customized content based on the treatment effect estimator.

16. The apparatus of claim 15, wherein:
the group selection component is further configured to generate a selection probability function for each of the plurality of users, wherein the treatment group and the control group are selected based on the selection probability function.

17. The apparatus of claim 15, wherein:
the group selection component is further configured to identify a user in the treatment group and the control group and to remove the user from the treatment group or the control group.

18. The apparatus of claim 15, wherein:
the group selection component is further configured to recursively partition the plurality of users based on the user feature vectors; and
the treatment group and the control group are selected based on the partitioning.

19. The apparatus of claim 18, wherein:
the partitioning is based on a Gram-Schmidt-Walk algorithm.

20. The apparatus of claim 18, wherein:
the group selection component is further configured to identify pairs of similar users among the plurality of users and to select a user from each of the pairs, wherein the partitioning is based on the selected user.
